(19) United States
(12) Patent Application Publication
(10) Pub. No.: US 2019/0307583 A1
(43) Pub. Date: Oct. 10, 2019

(54) Kinetic Sensing, Signal Generation, Feature Extraction, And Pattern Recognition For Control Of Autonomous Wearable Leg Devices

(71) Applicant: Massachusetts Institute of Technology, Cambridge, MA (US)

(72) Inventors: Hugh M. Herr, Somerville, MA (US); Roman Stolyarov, Bristol, RI (US); Luke M. Mooney, Westford, MA (US); Cameron Taylor, Cambridge, MA (US); Matthew Carney, Somerville, MA (US)

(21) Appl. No.: 16/347,666

(22) PCT Filed: Nov. 8, 2017
(86) PCT No.: PCT/US17/60710

§ 371 Date: May 6, 2019

Related U.S. Application Data
(60) Provisional application No. 62/419,192, filed on Nov. 8, 2016.

(51) Int. Cl. A61F 2/70; A61F 2/66; A61F 2/64; A61H 3/00; A61F 5/01; A61B 5/11; A61B 5/00; B25J 9/00

(52) U.S. Cl. CPC A61F 2/70; A61F 2/6607; A61F 2/64; A61H 3/00; A61F 5/0102; A61F 2002/607; A61B 5/6807; A61B 5/7275; B25J 9/0006; A61F 2002/6614; A61B 5/112

(57) ABSTRACT

An autonomous wearable leg device employs an array of sensors embedded along a support area, whereby a controller can generate a controlling command and send a controlling command to a prosthetic, orthotic, exoskeletal or wearable component to thereby control the prosthetic, orthotic, exoskeletal or wearable component. A method for controlling autonomous wearable device collects kinetic signals from an array of sensors embedded in a prosthetic, orthotic or exoskeletal component, wherein all values are extracted from at least one feature of the collected kinetic signals, which are applied to a controller that generates a controlling command that is sent to the prosthetic, orthotic exoskeletal component to thereby control the prosthetic, orthotic or exoskeletal component during a portion of a gait cycle.

This application claims the benefit of U.S. Provisional Application No. 62/419,192, filed on Nov. 8, 2016. The entire teachings of the above application are incorporated herein by reference.

### GOVERNMENT SUPPORT

This invention was made with government support under Grant No.: W81WH-14-C-0111 awarded by the U.S. Army Medical Research and Material Command. The Government has certain rights in the invention.

### BACKGROUND OF THE INVENTION

Autonomous wearable leg devices (AWLDs), which include autonomous transfemoral and transtibial prostheses, exoskeletons, and orthotics, must be designed to mimic biological behavior in order to be effective. However, currently there are deficiencies in both the control and mechanical design of such devices that prevent them from being biomimetic. Lower limb amputees experience several clinical problems while wearing conventional prostheses. These difficulties include deficient stability during standing and walking across a variety of terrains, asymmetric gait, slower walking speed, and increased metabolic rates when compared to people without amputations. Separately, individuals with lower limb pathologies or healthy individuals carrying heavy loads over extended distances often suffer from physical or mental fatigue. These difficulties have motivated the development of lower limb exoskeletons and orthoses to assist gait. [1] Recently, the development of autonomous exoskeletons has introduced the issue of control. In particular, an autonomous exoskeleton must effectively recognize the movement intent of its user and subsequently actuate a biomimetic control trajectory for the desired movement. However, despite the need for biomimetic control strategies and mechanical designs of AWLDs, the state of the art of such devices suffers from a lack of biomimicry across different terrains, gait events, and gait activities. Thus we present in this invention two novel control methods for autonomous wearable leg devices as well as a novel mechanical design of such a device.

### Control Methods

The design and implementation of control strategies is a significant obstacle in the development of effective AWLDs. [2] An AWLD control system (including sensors, control algorithms, and device actuators) must respond biomimetically to gait events, changes in gait activity, or changes in ground terrain, and it must do so regardless of the device user. One system currently employed on the commercialized BiOM T2 powered ankle foot prosthesis, relies mainly on integrated ankle torque estimation to both determine the gait phase and modulate control variables within each phase (for example power output during powered plantarflexion). The system allows for automatic, accurate, and punctual transitions between gait modes; it is integrated in that all sensors are contained within the ankle-foot enclosure; and it is generalizable across users, walking speeds, many ambient environmental factors, and, to a degree, terrain slope. [3] [4]

Control strategies for bipedal systems generally rely on the knowledge of the ground reaction force (GRF) (i.e. force of interaction between the foot and the ground) and zero moment point or, more broadly, the center of pressure (COP) [5]. In biomechanics, several control methods are available that emphasize the ankle-foot joint behavior to understand the dynamics of human balance have been proposed [6] [7] [8]. For these control schemes, ankle-foot movements are actively controlled to reposition the COP beneath the foot. By modulating the COP location, the ground reaction force can effectively be controlled, hence obtaining dynamic balance stability [8] [9]. In legged robotics, GRF and COP are generally measured with a series of sensors embedded in a robot's feet. In human biomechanics, standard measuring techniques for the GRF and COP have been restricted to a laboratory setting, where analysis tools, such as video motion capture and calibrated force platform systems, are available. Wearable gait analysis tools have been enabled that accurately estimate the GRF components and COP [10] [11] [12] [13]. Moreover, sensing technologies have also been implemented in prosthetic devices for gait studies and prosthetic assessment [14] [15] [16] [17].

Walking, the most common form of gait activity, includes tasks that can largely be classified by different types of walking surface geometry (terrain), and in particular flat ground walking and stair and ramp ascent and descent. It is well known that natural human lower limb biomechanics change significantly in response to varying terrains and thus deviate from level ground ambulatory control. [18][19] These changes can manifest during both the swing and stance phases of walking. Thus to ensure biomimetic performance, AWLDs must be adaptive to gait events, activities, and terrains.

Broadly, adaptation can be divided into two components: prediction or estimation of the desired gait activity or terrain, and actuation in response to this prediction or estimation. To-date, predicting gait activity or terrain in lower limb prostheses typically has employed sensor fusion techniques, in which information from a variety of sensing modalities (including inertial measurement, kinetic, optical or sonar distance measurement, and surface electromyography) has been extracted using pattern recognition to classify terrain [20] [21] [22] [23]. Inertial, kinetic, optical, or sonar measurement relies on sensors that do not directly communicate with the human. These sensors are thus more applicable to a user independent control system than surface electromyography, which exhibits significant variability depending on electrode positioning and both physiological state and constitution.

### Mechanical Design

The application of positive work to the environment is what enables locomotion. In human gait this comes in the form of torque applied about the joints, and their net energy is applied to the ground through the ankle and foot. A normative bodied human ankle applies 1.6±0.2 Nm/Kg of torque with 3±1 W/kg of power to the environment in normal walking [25]. A passive lower limb prosthesis is only able to provide as much energy as is stored during controlled dorsiflexion and knee flexion, leaving a deficit of roughly 0.2 J/kg of net work per step. This energy deficit at the ankle is compensated by increased work from other joints, such as increased swing from the hip. This puts additional load on these remaining joints, possibly leading to accelerated onset of osteoarthritis. Powered prosthetic devices have been shown the ability to return a more biologically accurate motion to a wearer's gait by applying torque at the knee [24] and also devices that apply torque at the ankle [26]. The powered prosthesis is able to control energy flow by absorbing negative work during dorsiflexion and applying positive work to the environment by converting stored electrical energy into plantarflexion torque at the powered joint.

Some work has begun in understanding how the foot and subtalar joints affect gait and stability. Level, flat ground walking requires primarily sagittal plane motion of knee and ankle joints, and inversion-eversion motions are generally afforded simply by compliance inherent in split-spring designed prosthetic foot devices. Powered inversion-eversion about the subtalar joint, however, may increase user stability in uneven terrain and in the presence of moving ground platforms such as the motions felt when standing on a subway or bus. Two other devices are known to have begun investigating the behavior of powered inversion-eversion devices, on a mobile system [27] and one a simulator system for experimentation [28]. Similarly, most prosthetic foot devices operate as a passive springs [29] or clutched passive devices [30]. A powered foot, or metatarsophalangeal may contribute to not only biological accuracy in gait as is defined by the LLTE [29], but also stabilization.

### SUMMARY

This invention is directed toward novel control algorithms and mechanical designs of autonomous wearable leg devices, which include autonomous transfemoral or transtibial prostheses, exoskeletons, and orthotics. More specifically, inventions include novel methods of detecting gait events, activities, and terrains in autonomous wearable leg devices in real time using kinetic or non-contact sensing modalities, and novel mechanical designs of autonomous leg prostheses with multiple degrees of freedom.

The invention generally is directed to an autonomous wearable leg device for integrated, real-time, and kinetic sensing, and to a method for controlling an autonomous wearable leg device.

In one embodiment, the invention is an autonomous leg device for integrated, real-time, kinetic sensing, including a prosthetic, orthotic or exoskeletal component that includes a support area. An array of sensors is embedded along the support area, and a controller is in communication with the array, whereby the sensors of the array can collectively sense and transmit spatially-dependent pressure signals to the controller, and whereby the controller can generate a controlling command and send the controlling command to the prosthetic, orthotic or exoskeletal component, and thereby control the prosthetic, orthotic, or exoskeletal component.

In another embodiment, the invention is a method for controlling an autonomous wearable device, including collecting kinetic signals from an array of sensors embedded in a prosthetic, orthotic or exoskeletal component of the autonomous wearable device worn by a subject during a portion of a gate cycle. Raw values of at least one feature are extracted from the collected kinetic signals, and then the feature is applied to a controller. A controlling command is generated by the controller. The controlling command is then sent to the prosthetic, orthotic or exoskeletal component to thereby control the prosthetic, orthotic or exoskeletal component during the portion of the gait cycle.

In still another embodiment, the invention is directed to an autonomous prosthetic, orthotic, or exoskeletal device that includes: an ankle frame; a pair of actuators, each actuator being mounted on the ankle frame and connectable to a power source and a control signal, wherein the actuators are independently controllable; a foot interface connected to the actuators, whereby actuation of either of the actuators transmits force to the foot interface; and at least one hinge at the frame linking the ankle frame to the foot interface, whereby synchronous movement of the actuators causes plantar flexion or dorsiflexion of the foot interface, and differential movement of the actuators causes eversion or inversion of the foot interface.

In another embodiment, the invention is a prosthetic foot that functions as a powered metatarsophalangeal joint. A mounting plate serves as a foot frame, and an actuator is mounted on the foot frame and connectable to a power source and a control signal. A hinge is linked to the foot frame, and a toe component is linked to the actuator, whereby the actuator causes the toe component to move relative to the hinge joint, thereby functioning as a powered metatarsophalangeal joint of the prosthetic foot.

In another embodiment, the invention is an autonomous prosthesis that includes an actuated knee component with a single actuated degree of freedom, an ankle component with two actuated degrees of freedom and linked to the actuated knee component, and a foot component with a single actuated degree of freedom linked to the ankle component.

In still another embodiment, the invention is an autonomous prosthesis that includes an actuated knee component with a single actuated degree of freedom, an ankle component with two actuated degrees of freedom and linked to the actuated knee component, and a passive foot component.

In another embodiment, the invention is an autonomous prosthesis that includes a passive knee component, an ankle component with two actuated degrees of freedom and linked to the passive knee component, and a foot component with a single actuated degree of freedom and linked to the ankle component.

In another embodiment, the invention is an autonomous prosthesis that includes a passive knee component, an ankle component with two actuated degrees of freedom and linked to the passive knee component, and a passive foot component linked to the ankle component.

In still another embodiment, the invention is a method for controlling an autonomous wearable leg device. The method includes using non-contact sensors integrated into a foot covering to detect or characterize obstacles or terrain changes in real time, and using the detection or characterization of obstacles or terrains to modulate a control algorithm of the autonomous wearable leg device in real time.

In yet another embodiment, the invention is a prosthetic device that functions as a powered metatarsophalangeal joint. In this embodiment, the invention includes a mounting plate that is a frame, an actuator mounted on the frame and connectable to a power source and a control signal, wherein the actuator is independently controllable. A hinge is linked to the frame, and an appendage is linked to the actuator, whereby the actuator causes the appendage to move relative to the hinge.

In another embodiment, the invention is an autonomous prosthesis that includes an ankle component with two actuated degrees of freedom, and a foot component with a single actuated degree of freedom and linked to the ankle component.

Consistent with the advantages conferred by the natural biomechanics of terrain transitions, integrated terrain-specific control methodologies in lower limb prostheses, according to the invention, can significantly reduce fall risk, the metabolic cost of walking, and the pain experienced at the residual limb.

More specifically, the invention enables punctuality (as the majority of information is available during stance), integration (e.g., into a prosthetic foot, foot cover, or transtibial load cell), and generalizability (e.g., since embodiments can rely on the emergent biomechanics of terrain transitions and signal quality does not vary depending on constitutive or state properties of the user). Pattern recognition on GRF and COP by the device and method of the invention, estimated from signals available during the stance phase of walking, also enables autonomy by introducing automaticity in combination with accuracy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic representation of an ankle foot complex free body diagram to estimate COP according to another embodiment of the invention.

FIG. 2A is a schematic representation of mode, task and state transitions, along with relevant transitions employed in kinetic sensing and control to obtain biomimetic control strategies for an autonomous wearable lug device of the invention.

FIG. 2B is a schematic representation of a high-level lower limb prosthesis controller with kinetic sensors suitable for use in one embodiment of the invention.

FIG. 3A is a sensel diagram configuration of sensors on a standard prosthetic foot cover as employed in one embodiment of the invention.

FIG. 3B is a side view of the prosthesis assembly with the foot cover shown in FIG. 3A.

FIG. 4 is a visualization of using non-contact sensing in a foot covering to predict stair ascent.

FIG. 5 is a visualization of using non-contact sensing in a downward orientation in a foot covering to predict stair descent.

FIG. 6 is a visualization of using non-contact sensing in a backward orientation in a foot covering to predict stair descent.

FIG. 7A is a side view of a multi-degree of freedom powered prosthesis attached to a subject in one embodiment of an autonomous knee-ankle-foot prosthesis of the invention.

FIG. 7B is a magnified anterior perspective view of the prosthesis of FIG. 7A.

FIG. 8A is a posterior perspective view of an ankle prosthesis of FIG. 7A, but with a passive foot element.

FIG. 8B is a posterior view of the prosthesis of FIG. 8A.

FIG. 8C is a side view of a two-degree of freedom powered ankle prosthesis of FIGS. 8A and 8B in partially disassembled form.

FIG. 9 is a posterior isometric view of the prosthesis of FIG. 8A, but with the foot frame having a powered metatarsophalangeal joint.

FIGS. 10A-10D are side views of a two-degree of freedom powered ankle prosthesis demonstrating plantar flexion, shown in FIGS. 10A-10B, as a right-side view of neutral orientation (FIG. 10A), right-side view of plantar flexion (FIG. 10B), magnified right-side view of neutral orientation (FIG. 10C), and a magnified right-side view of plantar flexion (FIG. 10D).

FIGS. 11A-11D are magnified and sectioned representations of the ankle-foot prosthesis of FIGS. 10A-10B in various modes of action, specifically plantarflexion (FIGS. 11C-11D), magnified right-side view of neutral position with section line (FIG. 11A), section view O-O showing linkage elements at neutral position (FIG. 11B), magnified right-side view of plantarflexion position with section line (FIG. 11C), section view P-P showing linkage elements at plantarflexion position (FIG. 11D).

FIGS. 12A-12D are views of a two-degree of freedom powered ankle prosthesis demonstrating inversion/eversion, shown in FIGS. 12A-12B, as a right-side view of eversion orientation (FIG. 12A), right-side view of eversion partially disassembled, magnified and sectioned (FIG. 12B), posterior view of eversion/inversion orientation (FIG. 12D), and a magnified posterior section view of eversion (FIG. 12C).

FIGS. 13A-13C are representations of the ankle-foot prosthesis. FIG. 13A is a posterior view of the prosthesis. The subtalar axis of rotation shown in section view of FIG. 13B. FIG. 13C is a detail of FIG. 13B.

FIGS. 14A-14D are representations of a powered metatarsophalangeal joint, illustrating a posterior perspective view (FIG. 14A), posterior view (FIG. 14B), right-side view of a neutral orientation of the MTP joint (FIG. 14C), and a right-side view of an extended MTP joint (FIG. 14D).

FIGS. 15A-15D are representations of another embodiment of a powered metatarsophalangeal joint, illustrating a posterior perspective view (FIG. 15A), posterior view (FIG. 15B), right-side view of a neutral orientation of the MTP joint (FIG. 15C), and a right-side view of an extended MTP joint (FIG. 15D).

FIGS. 16A-16D are representations of another embodiment of a powered metatarsophalangeal joint, a posterior perspective view (FIG. 16A), posterior view (FIG. 16B), right-side view of a neutral orientation of the MTP joint (FIG. 16C), and a right-side view of an extended MTP joint (FIG. 16D).

### DETAILED DESCRIPTION OF THE INVENTION

The inventions described here are directed toward both the control methods and mechanical design of autonomous wearable leg devices (AWLDs) including autonomous transtibial or transfemoral prostheses, exoskeletons, and orthotics.

### Control Methods

These inventions include methods of actuating AWLDs by modulating force or torque, power, position, velocity, work, or other control variables or provide feedback to the user in the form of mechanical or electrical stimuli. The methods of the invention are performed in real time and employ integrated, autonomous sensing, microprocessing, and actuation systems.

In one embodiment, the invention is a method for controlling an AWLD that incorporates integrated, real-time, kinetic sensing. Kinetic sensors are embedded either on an area for ground support belonging to the AWLD or as one or a plurality of load cells within a prosthetic thigh or shank. A controller is in communication with the sensors, whereby the sensors can collectively sense and transmit spatially-dependent pressure signals or localized forces and moments to the controller, and whereby the controller can generate a controlling command and send the controlling command to AWLD, and thereby control the AWLD.

In one embodiment, the controller of the AWLD uses the spatially varying pressure signals to estimate ground reaction force (GRF) and center of pressure (COP), and extracts features on these signals within the time window when the support area is in the ground, and inputs these features into a machine learning classifier to predict gait activity, gait event, or terrain of the next step. In this embodiment, the vertical component F_{Z} of the GRF is estimated as:

$${F}_{Z} = {\sum }_{i = 1}^{N} {p}_{i} \qquad ( 1 )$$

where pᵢ is the strain at each sensor i and N is then total number of sensels. Furthermore, COP components Cₓ and C_{y} can be estimated as:

$${C}_{x} = \frac{{\sum }_{i = 1}^{N} {p}_{i} {x}_{i}}{{F}_{Z}} \\ {C}_{y} = \frac{{\sum }_{i = 1}^{N} {p}_{i} {y}_{i}}{{F}_{Z}} \qquad ( 2 )$$

where xᵢ and yᵢ are the positions of each sensor i. Additionally, signals from individual sensors can also be used as input into the controller, whether for actuation in a particular gait state or for detecting and triggering state, task, or mode transitions.

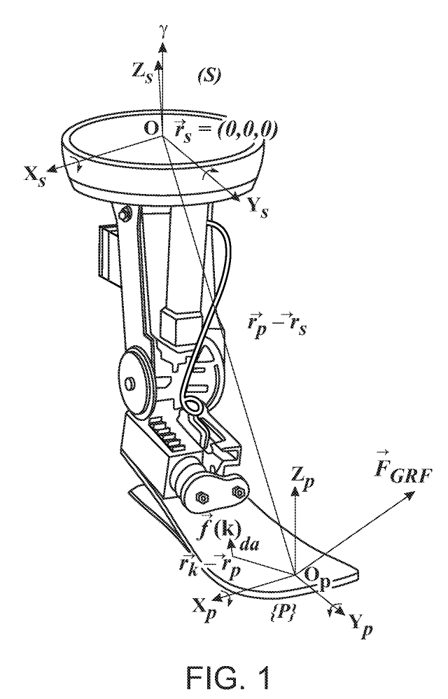

In the case of a transtibial load cell within a prosthesis, a static model can be used to estimate the GRF vector and location of the COP using three-dimensional moments and forces measured by such a load cell. The estimation contemplates only single stance support and the coordinate system employed for the model is Cartesian. The sign convention for the ground reaction force is positive upward and forward. The reference frames, shown in FIG. 1, employed are global {G}, load cell {S}, and support area {P}. Values measured by the load cell referenced can be mapped to forces in the corresponding COP frame {P}. COP location can be estimated with:

$${S}_{{r}_{COP {X}_{S}}} = {r}_{{PX}_{S}} = \frac{- {}_{}^{S}{M}{}_{{Y}_{S}}^{} - ( {r}_{{PZ}_{S}} {}_{}^{S}{F}{}_{{X}_{S}}^{} )}{{}_{}^{S}{F}{}_{{X}_{S}}^{}} \\ {S}_{{r}_{COP {Y}_{S}}} = {r}_{{PY}_{S}} = \frac{- {}_{}^{S}{M}{}_{{X}_{S}}^{} - ( {r}_{{PZ}_{S}} {}_{}^{S}{F}{}_{{Y}_{S}}^{} )}{{}_{}^{S}{F}{}_{{Z}_{S}}^{}} \qquad ( 3 )$$

Here the left superscripts represent the reference frame and the right subscripts represent the coordinate direction. The values employed are defined in the following way: ^{S}M_{YS}, ^{S}F_{YS} are the moment and force, respectively, around the Y_{S} axis of frame {S}; ^{S}M_{XS}, ^{S} F_{XS} are the moment and force, respectively, around the X_{S} axis of frame {S}; ^{S}F_{ZS} is the force in the Z_{S} direction of frame {S}; ^{r}P_{ZS} is the distance in Z_{S} to the COP. For an active prosthesis system this value can be computed as Z₀ cos γ where Z₀ is the load cell height at zero degrees of ankle flexion and γ is the shank angle in {G}. This assumes only rotation in the sagittal plane with no adduction or abduction in the joint. Assuming negligible inversion-eversion of the foot-ankle joint system, we can assume that the rotation matrix that relates frames {S} relative to {P} can be represented by this single sagittal angle γ. Given the COP location, the forces that interact with the ground, relative to the COP frame {P}, can then be expressed as:

^{P}F_{X}_{_{S}}=^{S}F_{X}_{_{S}}

^{P}F_{Y}_{_{S}}=^{S}F_{Y}_{_{S}} cos γ−^{S}F_{Z}_{_{S}}

^{P}F_{Z}_{_{S}}=^{S}F_{Y}_{_{S}} sin γ−^{S}F_{Z}_{_{S}} cos γ (4)

In the case of a transfemoral load cell, estimation of COP and GRF can be performed using knowledge of knee state and spatial transformations similar to those used for the transtibial load cell.

In another embodiment, a machine learning classifier can be used on the kinetic signals to predict upcoming gait events or terrains based on features extracted from these signals in real time. The classifier could include a heuristic method, a Naive Bayesian classifier; a decision tree classifier; a linear or quadratic discriminant classifier; a neural network classifier; and a logistic regression classifier.

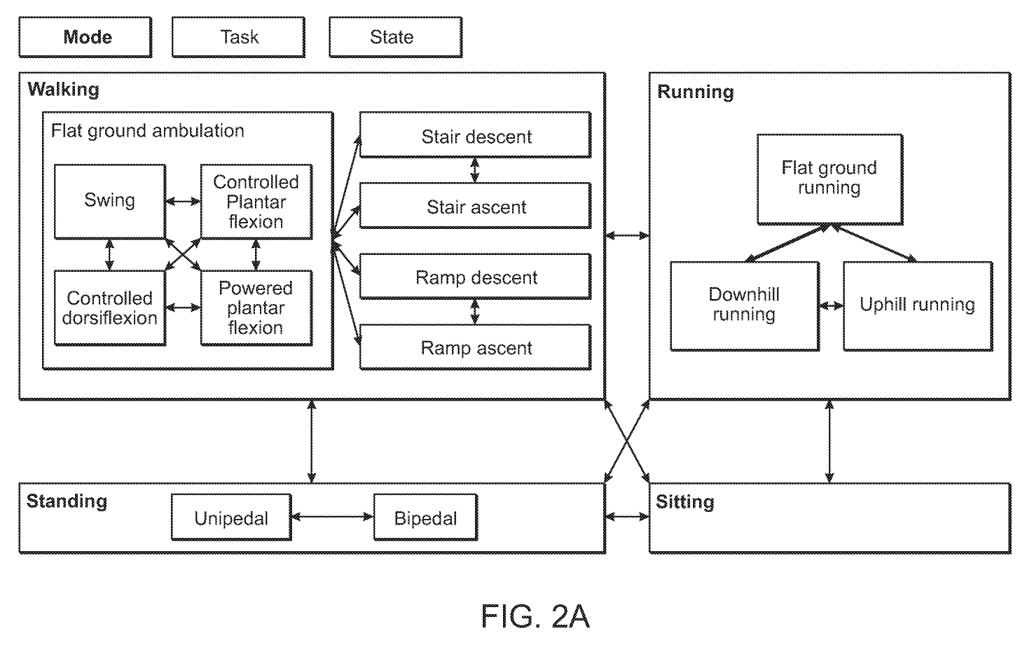

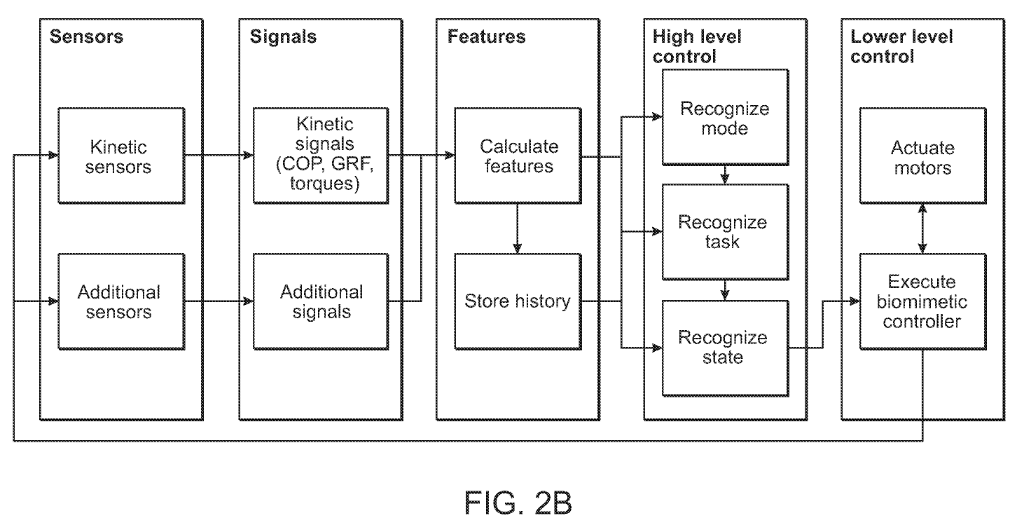

In an embodiment of the invention, a method includes application of kinetic sensing and control to obtain biomimetic control strategies for AWLDs to define a hierarchical control architecture. One level of such a hierarchy is mode, which includes such general activities as walking, running, sitting, or standing. Another level is task, the categories of which vary depending on activity. For example, walking tasks include walking on different terrains such as flat ground, stairs, or inclines. Finally, each task can be composed of a next hierarchical level, namely sequential states which are, typically, periodic. For example, walking on flat ground can be divided into swing phase, controlled plantarflexion, controlled dorsiflexion, and powered plantarflexion at the end of stance. Various modes, tasks, and states along with relevant transitions are visualized in FIG. 2A. A high-level control architecture is elucidated in FIG. 2B.

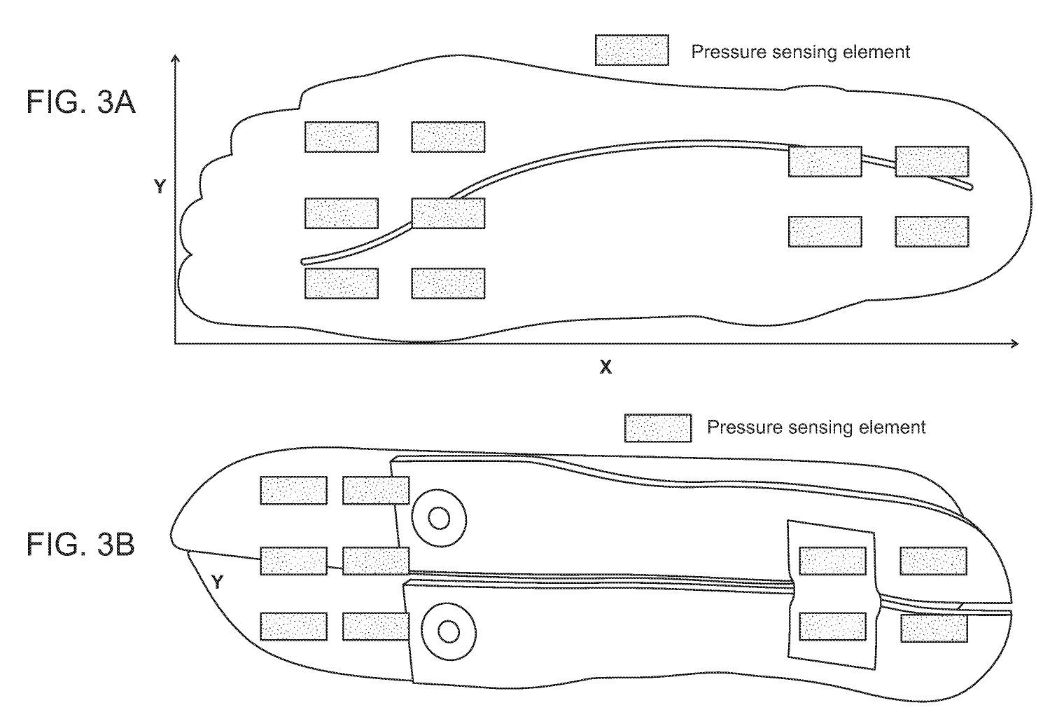

In one specific version of this device, an array of kinetic sensors can be embedded along the support area of a standard prosthetic foot cover and used to transmit spatially dependent pressure signals to the prosthesis controller. An example of such a configuration is exhibited in FIG. 3A.

Alternatively, an array of such sensors can be embedded within the support area of the prosthetic foot itself, as illustrated in FIG. 3B.

In yet another embodiment (not shown), an array of sensors is embedded within a transtibial or transfemoral prosthetic socket, or embedded within the sole of a shoe, or embedded within a sock.

In still another embodiment, the invention includes real-time provision of mechanical or electrical feedback to the user of an AWLD. Kinetic state variables, as described herein can be employed to provide feedback to the AWLD user in the form of mechanical or electrical stimuli. Mechanical stimuli can include vibration, application of normal pressure to the skin, skin pinching or strain application, or skin surface temperature variation. Electrical stimuli include electrical stimulation of muscles or nerves.

In another embodiment of the invention, one or more contact-free sensors can be employed within the foot covering of either a biological foot or prosthetic foot, either in addition or as an alternative to contact or pressure sensors. Examples of suitable contact-free sensors include cameras, distance-measuring sensors, and laser scanners. Such sensors, when employed, can be placed in communication with an AWLD controller that uses signals from these sensors to predict and respond to gait events and terrain changes by actuating AWLD in real time. The contact-free sensors can be aligned in any orientation or position either interior or exterior to an associated sock, foot cover, or shoe. With respect to powered exoskeletons and orthosis controllers, contact-free sensors can be aligned in any orientation or position either exterior or interior to an associated exoskeleton or orthosis.

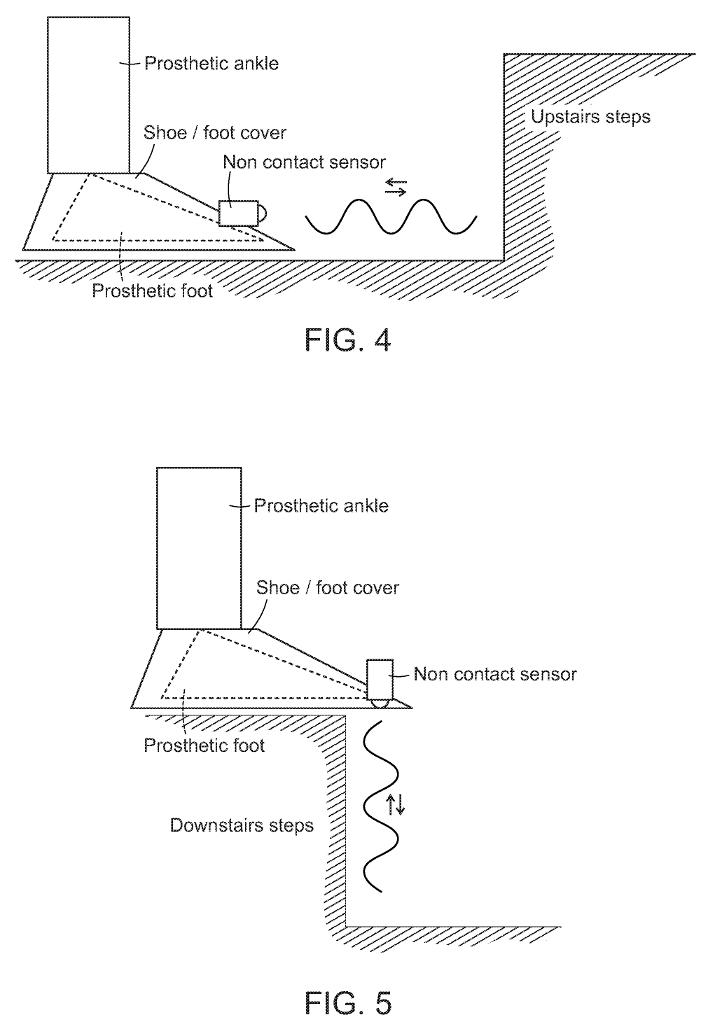

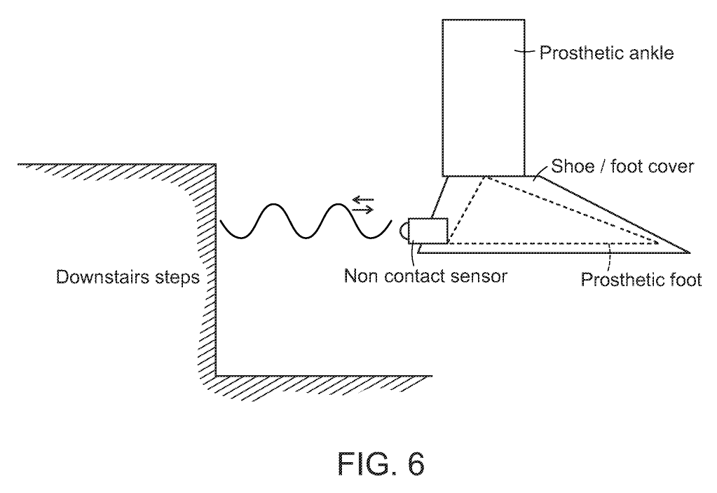

In one embodiment, at least one non-contact sensor is positioned in a forward orientation at a toe area of a shoe, whereby it is used to predict that the user will ascend stairs or clear an obstacle, as visualized in FIG. 4. In still another embodiment, at least one non-contact sensor is positioned in a downward orientation of the toe area of a shoe, whereby it is used to predict that the user will descend stairs when they position a lower extremity at the edge of a stair, as visualized in FIG. 5. In another embodiment, a non-contact sensor is positioned in a backward orientation at a back area of a shoe, whereby it is used to detect stair descent, as visualized in FIG. 6. In response to these predictions, a control algorithm is applied to actuate a prosthetic joint with a torque or position profile consistent with established biological norms for the predicted task or terrain.

Another embodiment of the invention includes real-time statistical or diagnostic monitoring in AWLDs using kinetic or non-contact sensors. Signals generated using these sensors can be monitored to provide statistical or diagnostic information about the AWLD. Statistical information can include, for example, statistics, step counts, information about power or force output, time spent, and electrical or metabolic work done in various modes, tasks, or states. This can be used to collect information from one user or across many users. Diagnostic information concerns any data regarding intended operation of the device or deviation from intended operation.

### Mechanical Design

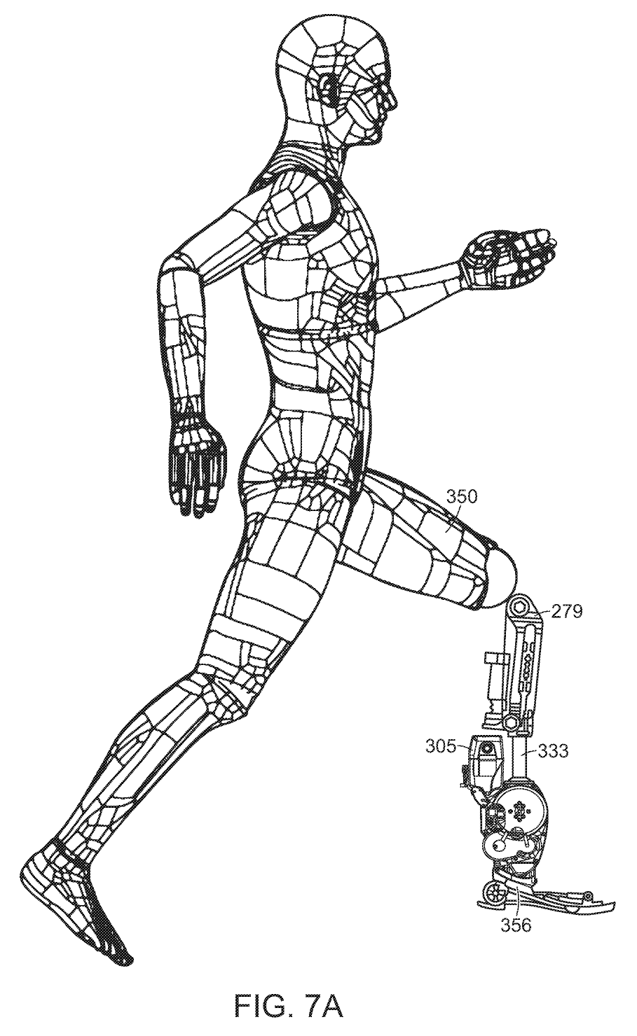

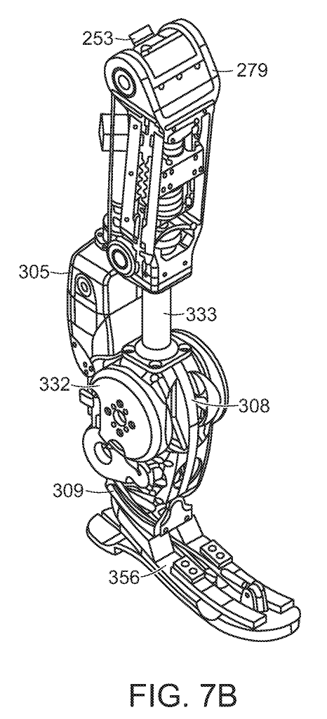

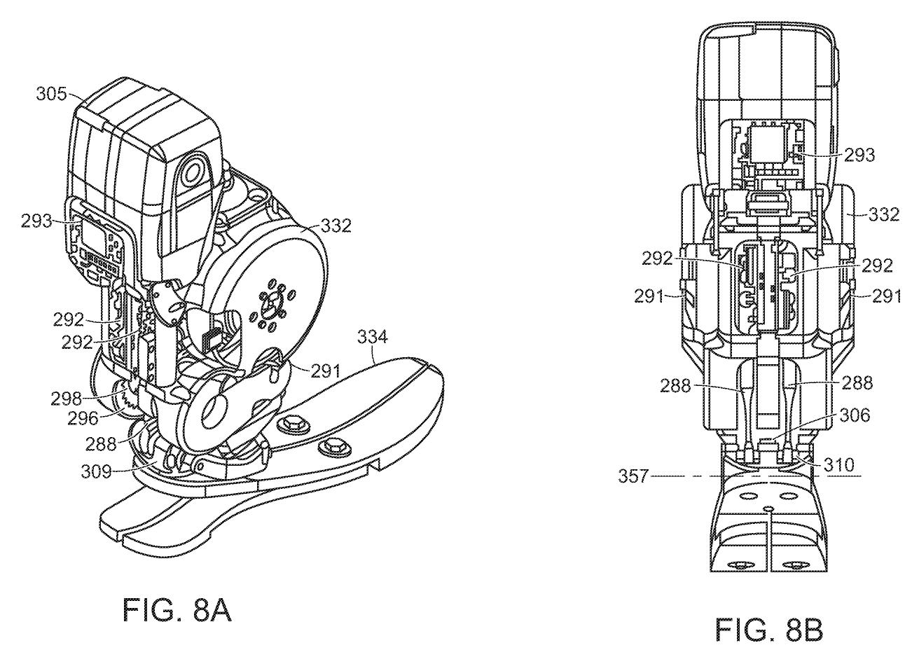

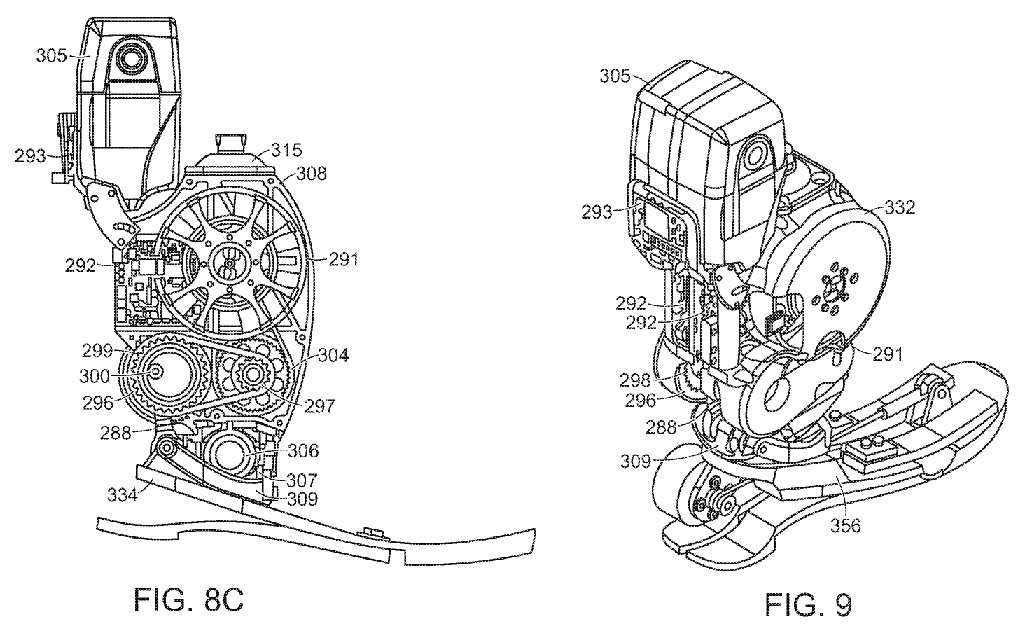

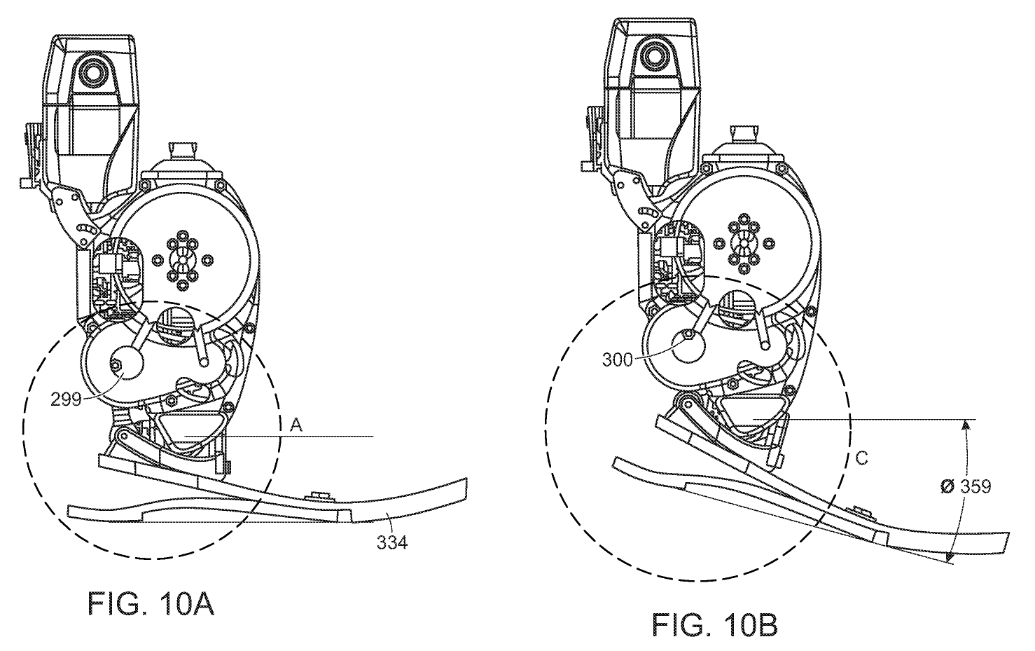

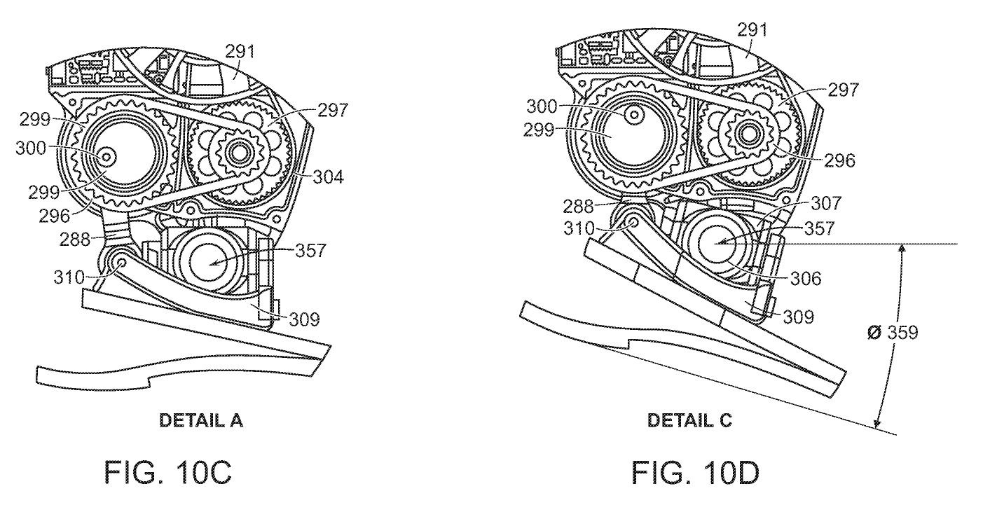

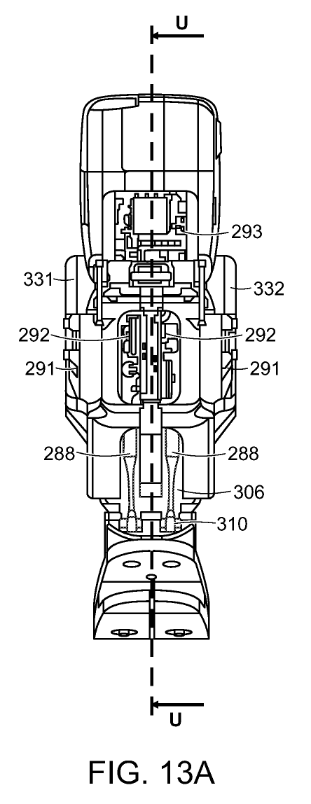

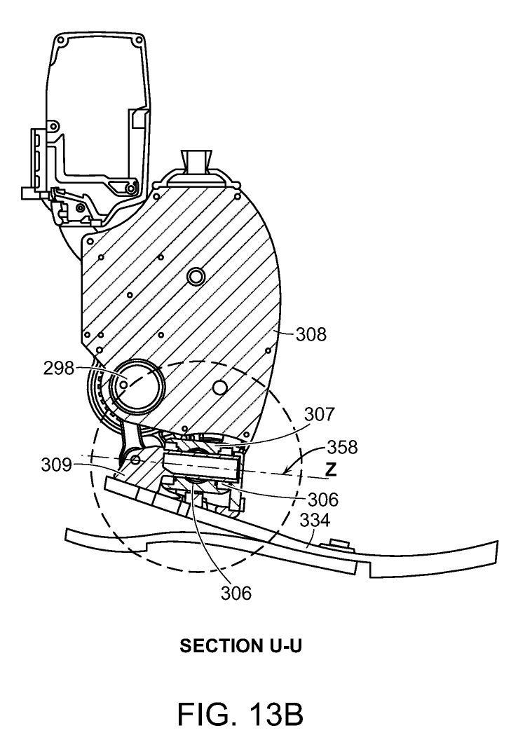

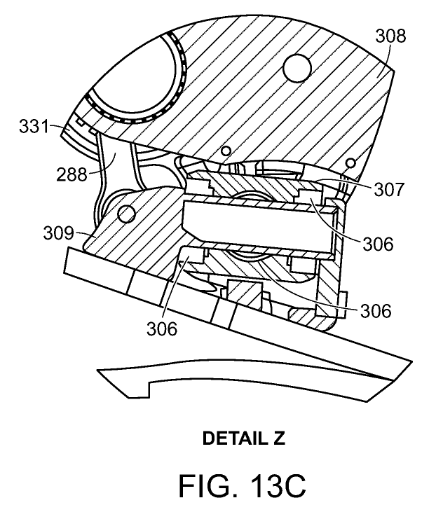

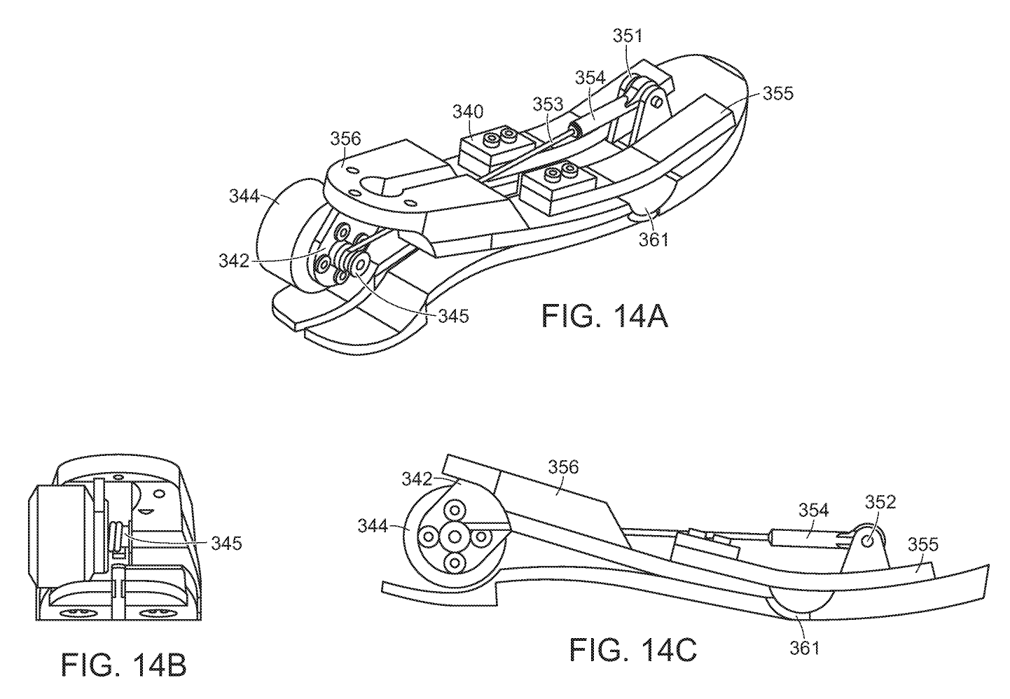

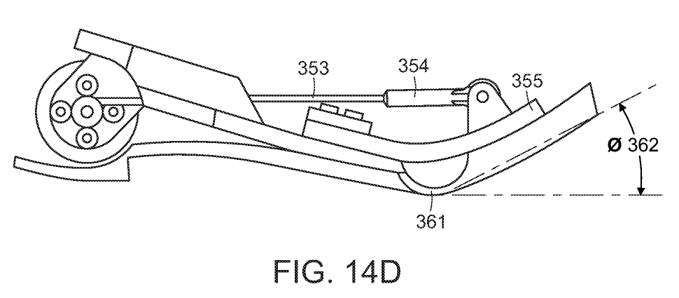

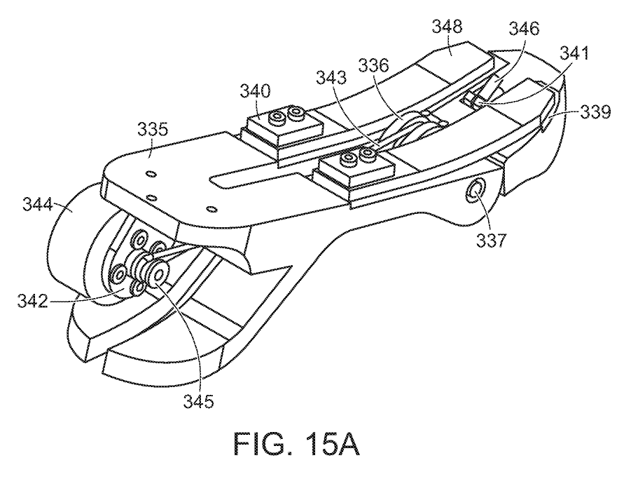

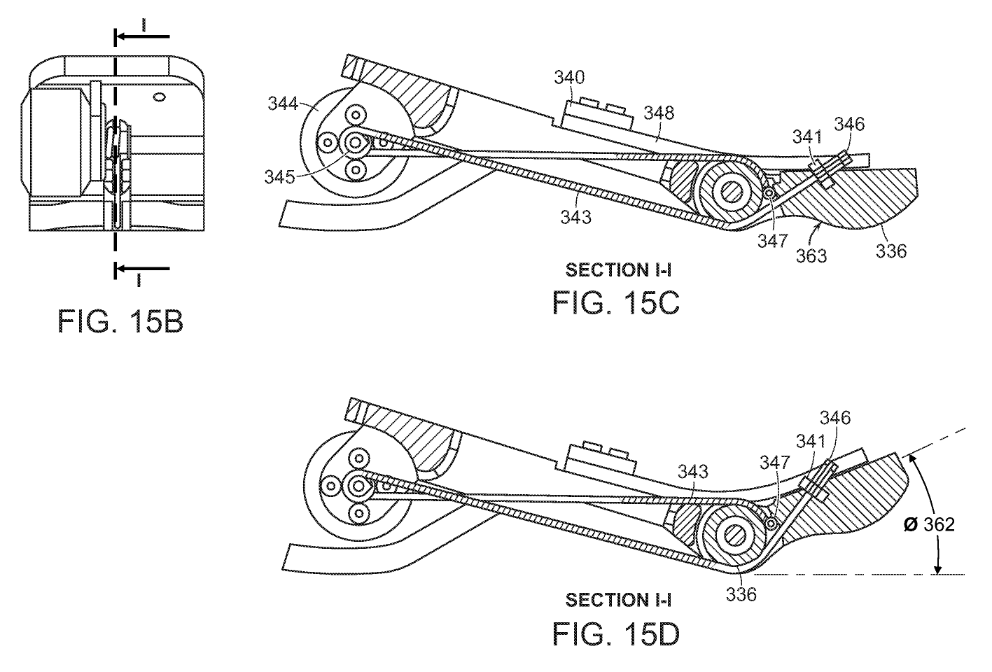

Another embodiment of the invention includes an autonomous multi-degree of freedom lower limb prosthesis, orthotic, exoskeleton, or wearable system that relies on signals, features, and pattern recognition on kinetic signals for control, such as an autonomous knee-ankle-foot, transfemoral prosthesis system with four powered/actuated degrees of freedom (DOFs) or powered axes of motion. The embodiment of the four DOF system shown in FIGS. 7A-7B connects to the user with a standard pyramid interface 253 and includes a knee prosthesis 279 with one DOF, connected with a pylon 333 to an ankle-foot (transtibial prosthesis) system with two DOF actuation 332, and a prosthetic foot with one DOF 356. In this embodiment there exists a single DOF at the knee 279, two DOF for ankle axis 357 shown in FIG. 10C, and subtalar axis 358 shown in FIG. 13B joints intersect at a differentially actuated gimbal 307 and there exists a metatarsophalangeal joint 356 and 337 in FIGS. 14-15. In all cases, the prosthesis will be powered by one or more, high energy density batteries 305 and controlled by an electronics platform 292 and 293 shown in FIGS. 8A-8C designed for bionic applications that include at minimum a microprocessor and motor driver functionality.

In this embodiment, an open-source FlexSEA bionic control architecture is utilized. This electronics architecture integrates high powered electronics 292 with flexible, high-fidelity sensing. In one embodiment, each actuated degree of freedom include power electronics 292 and high fidelity sensing electronics (referred to as “Execute boards”) controlled by Cypress Semiconductor PSOC microprocessors. Digital and analog input-output (I/O) functionality on the Execute board include native inertial measurement units, strain gage amplifiers, and expandable generic I/O. Generally, all DOFs are simultaneously connected and controlled with a single high level controller (Management board) 293, based on the STMicrosystems STM32 microprocessor, that fuses all sensors and state information. In this embodiment, the Management board 293 performs the high-level control that includes mode, task, state identification and operations as shown in FIG. 2, while the Execute motor drivers 292 perform low-level control such as motor current and torque control as well as sensor measurements. The flexibility of FlexSEA enables the rapid design and evaluation of a bionic system that requires high power control at a minimal size and mass.

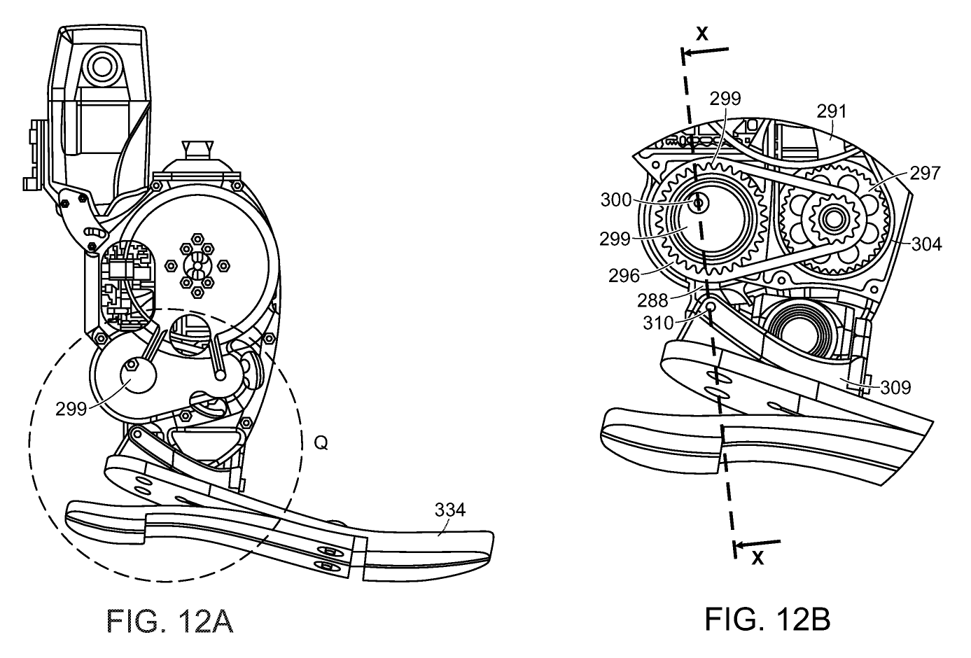

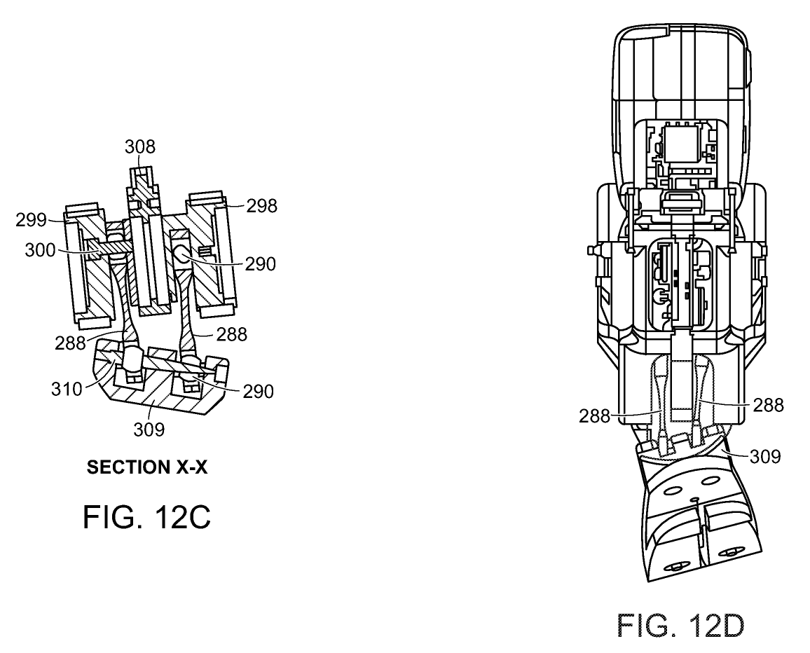

In a specific embodiment, the invention is an ankle-foot prosthesis, having two DOFs. A passive foot prosthesis is shown in FIGS. 8A-8B, 10-13 for simplicity. Alternatively, a powered metatarsophalangeal (MTP) foot could be attached to the foot frame 309. The term “foot frame” as employed herein is interchangeable with the term “foot interface.” In one embodiment, the two DOF axes intersect by way of a gimbal assembly 307 (shown unobstructed in FIGS. 8C, 10C-10D, 11C, 12B,12C and 13B, 13C). In the embodiment described herein this gimbal assembly operates as a differential; the configuration is actuated with a push-pull connecting rod linkage 288, and eccentric crank-arm 299 linkage assembly that are each components of two discreet but identical actuators—the components of one is shown with external structural covers 332 removed in FIG. 8C—that are mirrored across the sagittal plane of the prosthesis shown as section line U-U in FIG. 13A, and visible FIGS. 7-13. FIGS. 10A-10B show the ankle joint degree of freedom and FIG. 12D shows a rotation about the subtalar degree of freedom.

As that term is understood herein, the term “linkage” means a mechanical component that transmits bidirectional force with a push-pull action such as push-rod or flexure, a unidirectional tensile component such as a belt, cable, or chain, or a torsional component transmitting rotary motion directly through a rotary element, or a combination of elements such as a roller-cam element.

In detail, in this embodiment one actuator as shown in FIG. 8C is composed of the following components: a motor 291 with pinion pulley mounted on its shaft and coupled with a first timing belt 304 to an intermediate compound pulley 297 having both a larger and smaller pulley fused together such that driving the larger pulley causes rotation of the smaller pulley at the same angular velocity, and this smaller diameter pulley couples to another larger diameter pulley 299 by way of a second belt 296 forming a second stage gear-train, this last pulley then having a pin 300 mounted eccentric to the pulley center of rotation, and a pushrod 288 attached on one end to this eccentric pin and the other end to a pin 310 in the foot frame 309, and the motor drive electronics 292 convert battery 305 energy into controlled rotation of the motor. The posterior view of FIG. 8B and section line U-U in FIG. 13A shows this actuator is mirrored across the sagittal plane of the prosthetic device and mounted to the ankle frame 308 and both independently controlled actuators have kinetics and kinematics coordinated with the electronics of the motor drive 292 and/or the logic circuit board 293, all of which are powered by a battery 305 which itself may be composed of a plurality of energy storage cells. The motion of the two actuators are coupled together by way of the foot frame 309 that is connected to each independent push-pull rod linkages 288, and is grounded to the ankle frame 308 by way of a two-axis gimbal 307 that is both connected by rotational bearings 306 in FIG. 8B that form the subtalar degree of freedom axis 358 shown in FIG. 13B and serially connected to the ankle frame 309 through bearings 306 in FIG. 8C forming the ankle degree of freedom axis 357.

In the embodiment shown, high-torque, brushless, and direct current (BLDC) split phase sector motors (also referred to as “outrunner” motors) 291, for example, can be utilized as torque generators. In this embodiment, each actuator consists of one motor controlled by an Execute electronic control board 292, their synchronization controlled by a Manage microprocessor based high-level controller 293. Due to their torque-density, these split phase sector motors enable reduced transmission ratios from those of typically-available prosthetics. The lower reflected inertia and frictional losses of the reduced transmission ratio results in a higher bandwidth, more dynamic, efficient and quiet system, and also enables a more accurate observation of output torque by way of current sensing at the motor power electronics, reducing the need for additional load-cells, specific series compliance and displacement measurement systems.

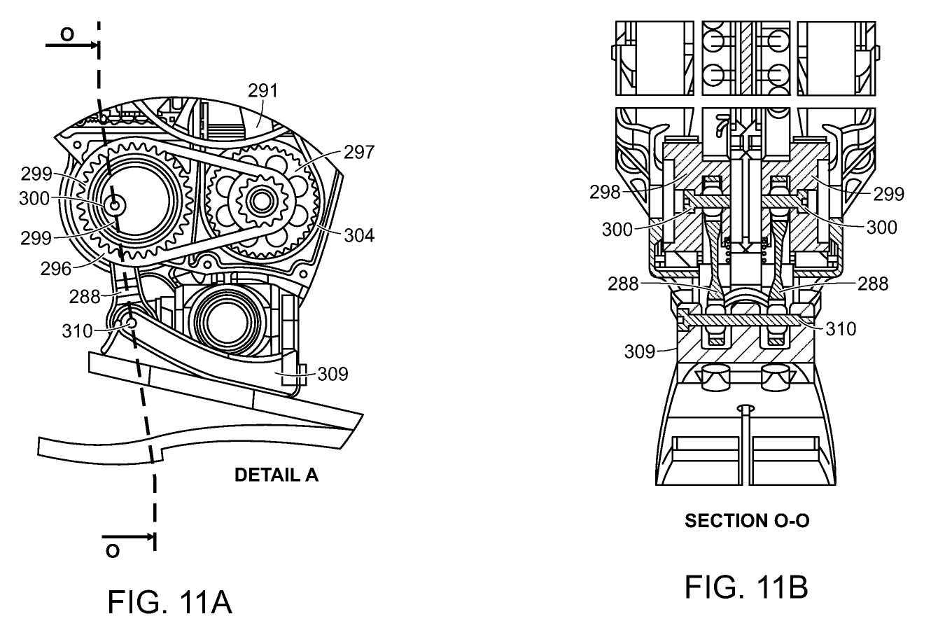

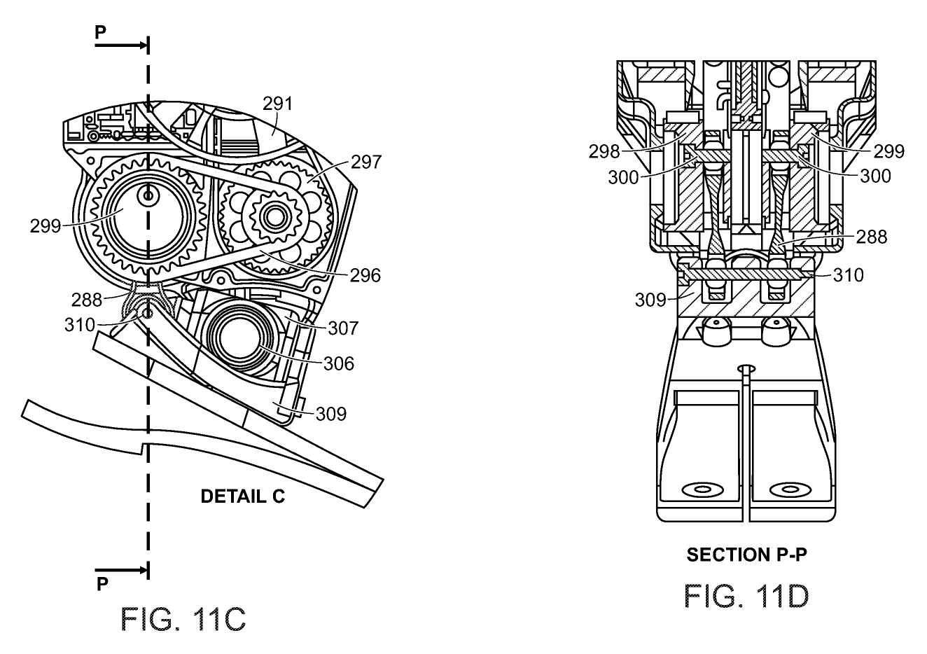

In this embodiment, homodirectional or synchronous motion between the left and right push-rods 288 affect ankle rotation for dorsiflexion and plantarflexion (plantarflexion is shown in FIG. 10B, 10D, 11C-11D), while differential displacement or asynchronous motion between left and right push-rods 288 affect inversion and eversion (FIG. 12A-12D). For example, this means if both motors move at the same speed in the same direction as viewed from the side, the foot frame will rotate about the ankle axis and not the subtalar axis, if the motors move in the same direction but at different speeds there will be both ankle rotation and subtalar rotation, and if either one motor remains stationary while the other moves or the motors move in opposite directions at any speed there will be subtalar and not ankle rotation. A neutral orientation for the ankle joint is set in the control software as a comfortable standing orientation for the ankle joint. For the purposes of description this will be considered the point when the fore and aft ball of the foot or equivalent components of the foot prosthesis are both touching level flat ground, this is assumed to be when the eccentric pin 300 is aligned horizontally with the center of rotation of the crank-pulley 299. The magnified view in FIG. 10C shows a neutral orientation as described. In this embodiment a clockwise rotation of both motors as seen from side view FIG. 10B will cause intermediate compound gear 297 to also rotate clockwise and crank pulley 299 will similarly rotate clockwise due to the timing belt coupling each of the gear stages together. The magnified view in FIG. 10D shows that foot frame 309 has been rotated clockwise about ankle axis 357 an angle 359 because pin 300 has rotated clockwise, now to a position nearly vertical with the axis of rotation of crank pulley 299, and the push-pull rod 288 has thus lifted the aft portion of the foot frame 309 with respect to the ankle joint bearing assembly 306 of gimbal 307, that grounds the foot frame to the ankle frame 308. FIG. 11A-11D show section views of the plantar flexion motion, where FIG. 11B shows a section cut viewed from the posterior and sectioned as shown by section line O-O in FIG. 11A. In FIG. 11B it can be seen that crank pulley 299 is mirrored across the sagittal plane to a secondary crank pulley 298, each of which independently support eccentrically located pins 300 that support push-pull rods 288 that are secured to a shared pin 310 attached to foot frame 309. In the neutral position as defined pins 300 are aligned horizontally with the axis of rotation of pulleys 298 and 299. In FIGS. 11C-11D plantarflexion is shown whereby mirrored and synchronous motion of the motors and ultimately crank pulleys have lifted the pins 300 to above the axis of rotations of pulleys 298, 299, lifting above the ankle axis of rotation 357 the shared pin 310 that is grounded to the aft portion of the foot frame 309, causing plantarflexion motion of the foot frame with respect to the ankle axis 357 that runs concentrically between the ankle joint support bearings 306.

For this embodiment, FIGS. 12A-12D demonstrate eversion/inversion motion of the subtalar axis 358 FIG. 13B, 13C. In the case of the foot frame moving about the subtalar joint, an inversion or eversion of the foot, the motors rotate asynchronously. FIG. 12A shows a subtalar rotation that for this example is called inversion as we take this to be a left leg prosthesis. The magnified view FIG. 12D shows the cover 332 removed and a section line X-X passing through the pin 300 and 310. Due to the asynchronous and rotational nature of this joint the section view in FIG. 12C shows partially sectioned pins 300 and 310. For subtalar joint motion the motors 291 of each actuator on the medial and lateral sides of the prosthesis move at different speeds and/or directions, by way of the belt drivetrain. If for example the lateral motor remains stationary, and the medial motor rotates clockwise the crank pulley, as shown in section view FIG. 12C, will raise the eccentrically located pin 300, thereby lifting the medial push-pull rod 288, lifting the medial side of the coupling pin 310 and, causing an inversion rotation about the subtalar axis by rotating the foot frame in the support bearings 306 (FIG. 8B) mounted in the gimbal 307 (FIG. 8C). Similarly, both crank pulleys could be rotating in opposite directions causing an inversion or eversion motion. Finally, for combined rotation about both the ankle and subtalar axes, both motors could be, for example, be moving in the same direction but at different speeds, causing the crank pulleys to lift the push-pull connecting linkage rods to rise at different speeds, leading to both a rotation about the ankle and subtalar joints, simultaneously.

The embodiment of the gimbal as described is more clearly visible in FIG. 13C. The gimbal 307 is a component that enables the intersection of more than one degree of freedom by way of coupling rotating elements into a single set of components. In this embodiment two axes intersect orthogonally, with a central component 307 supporting two coaxial bearings on each axis. The FIG. 13C section view cuts along a plane that intersects the subtalar axis at this instance. Bearings 306 for the subtalar axis are shown mounted to gimbal 307 and the shaft of foot frame 309. One of two bearings 306 of the ankle axis is partially visible behind the subtalar shaft. This bearing is housed in the gimbal and the shaft of this axis is split on either half and grounds the gimbal to the ankle frame 308 by way of the outer cover 331 and 332 (FIG. 13A).

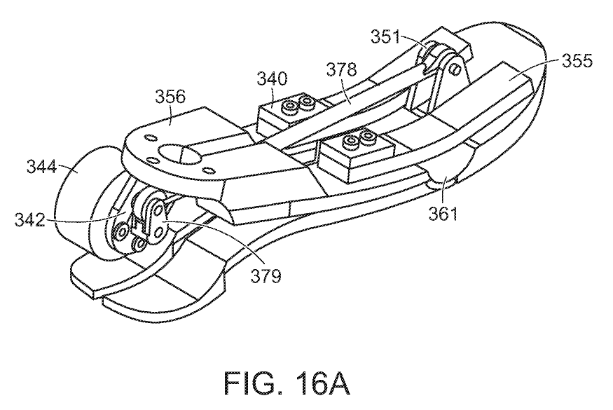

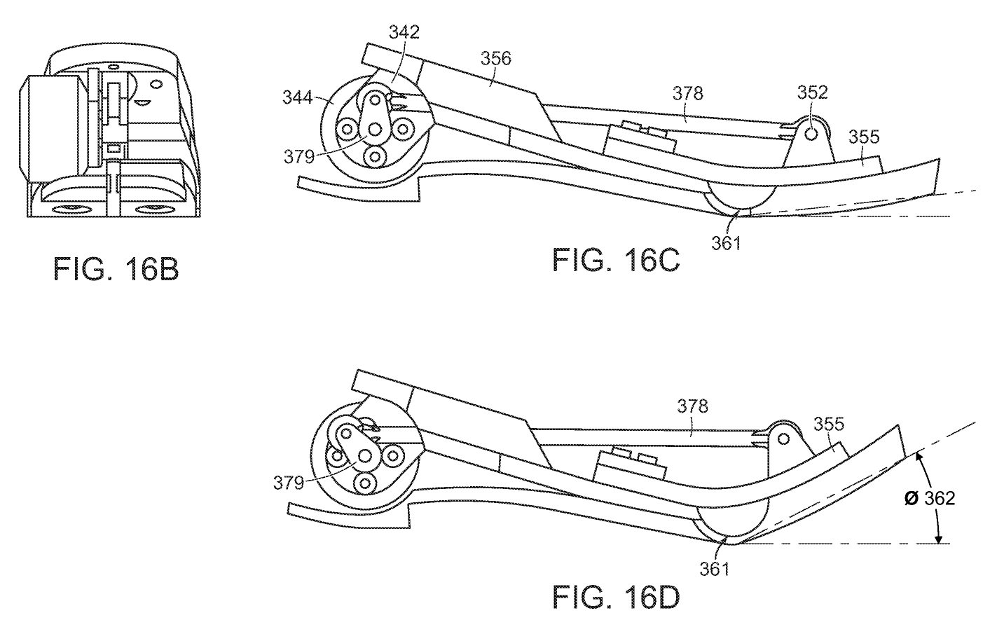

In still another embodiment, the invention includes a single DOF foot device (FIGS. 14-16) that can be employed to provide increased push-off, touch-down, balance and range of motion for a powered lower limb prosthesis. The similar biological counterpart of the actuated joint is the metatarsophalangeal joint. In FIGS. 14A-14D the combination of motor 344, capstan 345, tensile member 353 and end-fitting 354 act as a force actuator that applies a moment by way of the moment arm 351 about the DOF at the axis of rotation 361 for the powered metatarsophalangeal joint. In a similar embodiment shown in FIGS. 16A-16D the combination of motor 344, crank-arm 379, push-pull rod 378 act as a force actuator that applies a moment by way of the moment arm 351 about the DOF at the axis of rotation 361 for the powered metatarsophalangeal joint. In these embodiments torque is generated about the DOF by the elastic restoring force of a flexural hinge 361 (akin to a leaf-spring) whose return to its minimum energy (neutral) or resting position applies force to the external environment and provides energy for push-off. In the FIG. 14 embodiment, during gait phases the hinge is preloaded by a motor 344, that is mounted to the foot frame 356 by way of a motor mount 342, the motor shaft has a capstan 345 affixed that could just as well be a timing pulley and tensile strand 353 and push-off force is modulated by regulation of the length of the tensile strand. In the embodiment shown in FIGS. 16C-16D the end-effector push-off force profile is modulated by regulation of the position of the output moment arm 351 by way of the motor 344 rotating the crank arm 379, pushing or pulling the push-pull connecting rod 378. In the case where a larger joint stiffness is desired than that provided by the hinge further stiffness may be provided by the addition of parallel stiffness springs 355. In this embodiment these are cantilever beams that the MTP joint displaces when moved through the joint range of motion 362. FIGS. 14A-14D show a force actuator with additional parallel stiffness provided by one or a plurality of cantilever beams 355. These beams are fixed to the foot frame by using screws to affix a force distributing clamp 340. The axis of rotation 361 can be a pivot or a flexural hinge as shown in the FIG. 14D extending an angle 362. The hinge may not necessarily be flexural but could also be explicitly defined with a hinge or linkage assembly (for more complex motion profiles), a hinge is shown in another embodiment in FIGS. 15A-15D, with energy storage provided by distinct series or parallel elastic members 355. Another embodiment may be actuated with a crank arm, push-rod assembly, a tensile element, screw drive, linkage mechanism, or an actuator located directly at the joint to provide flexion and extension torque about the joint with additional torque regulation performed by modulating the preload against the parallel elastic element(s).

In yet another embodiment, in FIGS. 15A-15D the combination of motor 344, capstan 345, tensile member 343 and end-fittings 347, 346, 341 act as a force actuator that applies a moment about the pivot joint 337 by pulling tension on the output toe 336 at a distance away from the pivot axis 337, enforced by the geometry of 336 about the pivot. The section views FIGS. 15C-15D show the geometry in this embodiment to be circular, but it could just as well be a geometry tuned to match the desired torque characteristics of the joint, providing a cam type of behavior. In this embodiment torque is generated about the DOF bi-directionally by routing a tensile member 343 around a capstan 345 and affixing it to end points in the toe 336. One end of the tensile member has a passive end-fitting 347, the other end has means to tension the tensile member as necessary with components 341 and 346. In this embodiment the tensioning components include an end-fitting that is affixed to the tensile member and has external threads, around which an internally threaded nut-like components 341 is threaded until it engages with the toe 336 and further turning of the element causes elongation of the tensile member creating tension in the cable to provide friction at the capstan. The concave surface shape 363 of the toe 336 provides a specific point for the output force of the MTP joint to act on the environment. FIG. 14 shows an MTP joint without this feature, in that case the output force that can be applied to the environment is limited by the short distance from the point of contact to the pivot. In this case of surface geometry 363 a minimum distance at which a force can be applied on the environment is enforced, enhancing the effectiveness of the applied torque at the joint. In the case where a larger joint stiffness is desired than that provided by the motor driven components, further stiffness may be provided by the addition of parallel stiffness springs 348. In this embodiment these are cantilever beams that the MTP joint displaces when moved through the joint range of motion 362. FIGS. 15A-15D show a force actuator with additional parallel stiffness provided by one or a plurality of cantilever beams 348. These beams are fixed to the foot frame by using screws to affix a force distributing clamp 340, their free end is loaded against a low friction material 339, something such as a PTFE, or acetal polymer may suffice. The axis of rotation 337 can be a pivot or a flexural hinge as shown in the FIG. 14D and can extend and flex with an angle 362.

In FIGS. 16A-16D the combination of motor 344, crank-arm 379, push-pull rod 378 act as a force actuator that applies a moment by way of the moment arm 351 about the DOF at the axis of rotation 361 for the powered metatarsophalangeal joint.

One embodiment of the invention is an autonomous four DOF knee-ankle-foot prosthesis system comprising a knee joint with a single actuated DOF, ankle-foot-prosthesis with two actuated DOFs, and prosthetic foot with single actuated DOF. Another embodiment includes: an autonomous three DOF knee-ankle-foot prosthesis system comprising a knee with a single actuated DOF; an ankle-foot prosthesis with two actuated DOFs; and a passive prosthetic foot. Still another embodiment of the invention is an autonomous single DOF knee-ankle-foot prosthesis that includes: a knee with a single actuated DOF; a passive ankle-foot prosthesis; and a passive prosthetic foot. Yet another embodiment of the invention is an autonomous three DOF knee-ankle-foot prosthesis that includes: a passive knee joint; an ankle-foot prosthesis with two actuated DOFs; and a prosthetic foot with one actuated DOF. Still another embodiment of the invention is an autonomous two DOF knee-ankle-foot prosthesis that includes: a passive knee joint; an ankle-foot prosthesis with two actuated DOFs; and a passive prosthetic foot. In another embodiment, the invention is an autonomous three DOF ankle-foot prosthesis that includes: an ankle-foot prosthesis with two actuated DOFs; and a prosthetic foot with one actuated DOF. Another embodiment is an autonomous two DOF ankle-foot prosthesis that includes: an ankle-foot prosthesis with two actuated DOFs and a passive prosthetic foot.

The following is a description of a demonstration of select embodiments of the invention, and is not intended to be limiting in any way.

### EXEMPLIFICATION

We performed a preliminary study in which we asked six subjects with unilateral transtibial amputations (ages ranged between 28 and 66, heights 1.68 m and 1.90 m, and weights between 130 and 229 lbs, all K4 ambulators) to traverse various terrains while signals were collected from an array of kinetic sensors embedded in an insole on the side of the prosthetic device.

Data for each subject was collected in several trials each involving between eight and twelve circuits. In each trial, subjects were asked to undergo several circuits involving transitions to and from a staircase and flat ground while wearing a powered transtibial prosthesis aligned to a custom fitted socket via a pylon of appropriate length. Each circuit comprised one complete ascent, turn-around maneuver, descent, and subsequent turn-around, allowing for transitions to and from the terrain in either direction. Stairs terminated in a platform allowing for approximately one complete gait cycle completion after exiting the staircase and before turning around.

Each trial was conducted such that the subject was visible by at least four motion capture cameras at all times, which were set to a capture rate of 100 Hz. Data were collected from the prosthesis sensors (including an inertial measurement unit and motor and ankle joint encoders) and from resistive pressure sensing insoles developed by Tekscan. All subjects wore a rubber cosmesis over their carbon fiber foot, with pressure sensing insoles positioned between the cosmesis and shoe insole and taped to the latter. Pressure sensors were cut by hand to match the size of the shoe's insole.

Subjects were asked to begin each trial with a quiet period of static, bilateral stance to establish a reference pressure distribution on the sensor. Additionally, sensors were calibrated using a proprietary method provided by Tekscan software that involved collecting a short trial of unilateral (one-legged) stance on the instrumented leg. All subjects were physically labeled by a full lower body marker set (described in a further section).

Subjects used the BiOM ankle-foot prosthesis (FIGS. 12A and 12B) to complete circuits for all trials. This device executes a controller for level ground walking tuned to the needs of every subject. The device controller divides walking into five major states, including early and late swing (position control for ground clearance), early stance (impeded plantarflexion), late stance (impeded dorsiflexion), and late stance power (powered plantarflexion). Tuning parameters of greatest diversity across subjects include the magnitude and timing of torque delivery during powered plantar flexion at different walking speeds, and all were set to optimize symmetry and subject-reported comfort, and set for the duration of the experiment for each subject.

Pressure sensing insoles were part of the F-Scan In-Shoe Analysis System developed by Tekscan. The sensors were originally size 14 (US) and trimmed in accordance with the foot size of each subject. Sensor technology was resistive, with 0.15 mm thickness, 25 sensel per in2 resolution, and 862 KPa pressure range. An example is displayed in FIG. 13. A 12-camera Vicon 8i motion capture system was used in the study to label all terrain transitions encountered in the trials.

For each trial, various time varying signals were extracted from the frame data including the centers of pressures, integrated pressures across the ball, heel, and whole foot, and derivatives of these signals. All integrated pressure signals were normalized by the mean maximum value achieved across all flat ground to flat ground steps. Next, all stance phase periods within the trial were identified using a threshold on total integrated force, and the boundaries of each stance window were used to identify foot contact and foot off, respectively. For each stance period, a terrain (either flat ground, upstairs, or downstairs) was defined using motion capture data, which included data about subject and staircase position.

We then extracted various features for each signal from only the stance phase, including mean, maximum, minimum, range, standard deviation, and initial and final values. Additionally, the initial length of stance was used as a feature. All features were then standardized to zero mean and unit variance across the entire dataset.

Finally, we employed pattern recognition on the features of each step to predict the labeled terrain of the next step correctly. We were able to attain an accuracy of approximately 86% using a 20-fold cross-validated linear discriminant analysis classifier with empirical prior probabilities on data containing transitions among flat ground, upstairs, and downstairs steps. Complete data for all feature subsets using empirical priors are presented in the FIG. 18. Error at 0 features indicates the theoretical expected error of guessing using only the prior distribution.

### REFERENCES

1. Mooney, Luke M., Elliott J. Rouse, and Hugh M. Herr. “Autonomous Exoskeleton Reduces Metabolic Cost of Human Walking.” Journal of NeuroEngineering and Rehabilitation J NeuroEngineering Rehabil 11.1 (2014): 151.
2. Dollar, Aaron M., and Hugh Herr. “Lower Extremity Exoskeletons and Active Orthoses: Challenges and State-of-the-Art.” IEEE Trans. Robot. IEEE Transactions on Robotics 24.1 (2008): 144-58.
3. Grabowski A, Herr H. “Bionic ankle-foot prosthesis normalizes walking gait for persons with leg amputation.” Proc Biol Sci (2012).
4. M. F. Eilenberg, H. Geyer, and H. Herr, “Control of a Powered Ankle Foot Prosthesis Based on a Neuromuscular Model,” IEEE Trans. Neural Syst. Rehabil. Eng. IEEE Transactions on Neural Systems and Rehabilitation Engineering, vol. 18, no. 2, pp. 164-173, 2010.
5. Vukobratovic, Miomir, and Davor Juricic. “Contribution to the Synthesis of Biped Gait.” IEEE Transactions on Biomedical Engineering IEEE Trans. Biomed. Eng. BME-16.1 (1969): 1-6.
6. Winter, David A., Aftab Patla, and Francois Prince. “Stiffness Control of Balance during Quiet Standing.” Gait and Posture 5.2 (1997): 154-55.
7. Balasubramaniam, Ramesh, and Alan M. Wing. “The Dynamics of Standing Balance.” Trends in Cognitive Sciences 6.12 (2002): 531-36.
8. B., Marko, and Hugh Herr. “Ground Reference Points in Legged Locomotion: Definitions, Biological Trajectories and Control Implications.” Mobile Robots: Towards New Applications (2006).
9. Popovic, M. b., and H. Herr. “Global Motion Control and Support Base Planning.” 2005 IEEE/RSJ International Conference on Intelligent Robots and Systems (2005).
10. Veltink, P. H., Liedtke, C., Droog, E., Van der Kooij, H., 2005. “Ambulatory measurement of ground reaction forces.” IEEE Transactions on Neural Systems and Rehabilitation Engineering. Vol 13. No. 3. pp. 423-427.
11. Lawrence, T., Schmidt, L., R., N., 1997. “Wireless In-Shoe Force System.” Proceedings 19th IEEE Int. Conf. Engr. in Medicine and Biology Society. Chicago, Ill. pp. 2238-2241.
12. Davis, B. L., Perry, J. E., Neth, D. C., and Waters, K. C. 1998. “A device for simultaneous measurement of pressure and shear force distribution on the plantar surface of the foot.” Journal of Applied Biomechanics. Vol. 14. pp. 93-104.
13. Morris, S. J., Paradiso, J. A., 2002. “Shoe-integrated sensor for wireless gait analysis and real-time feedback.” Proceedings of the Second Joint EMBS/BMES Conference. October. pp. 2468-2469.
14. Berme, N., Lawes P., Solomonnidis S., and Paul J. P. “A shorter pylon transducer for measurement of prosthetic forces and moments during amputee gait.” Eng Med. 4. pp. 6-8.
15. Morimoto, S. “Evaluation of foot ankle units by an ambulatory gait measuring system.” Proceedings of the 7th World Congress of the International Society for Prosthetics and Orthotics (ISPO). Chicago. Vol. Il. pp. 323
16. Sanders, J. E., Miller, R. A., Berglund, D. N., Zachariah, S, 1997. “A modular six-directional force sensor for prosthetic assessment: a technical note.” Journal of Rehabilitation Research and Development. Vol. 34. no. 2. pp. 195-202.
17. Takahashi, Y., Kagami, S., Ehara, Y., Mochimaru, M., Takahashi, M., Mizoguchi, H., 2004. Six-axis force sensing footwear for natural walking analysis. IEEE International Conf. on Systems, Man and Cybernetics. pp. 5374-5379.
18. Gates, D. h., J. Lelas, U. d. Croce, H. Herr, and P. Bonato. “Characterization of Ankle Function during Stair Ambulation.” The 26th Annual International Conference of the IEEE Engineering in Medicine and Biology Society (2004).
19. Fradet, Laetitia, Merkur Alimusaj, Frank Braatz, and Sebastian I. Wolf “Biomechanical Analysis of Ramp Ambulation of Transtibial Amputees with an Adaptive Ankle Foot System.” Gait and Posture 32.2 (2010): 191-98.
20. Hargrove, Levi J., Aaron J. Young, Ann M. Simon, Nicholas P. Fey, Robert D. Lip-schutz, Suzanne B. Finucane, Elizabeth G. Halsne, Kimberly A. Ingraham, and Todd A. Kuiken. “Intuitive Control of a Powered Prosthetic Leg During Ambulation.” Jama 313.22 (2015): 2244.
21. Yuan, Kebin, Qining Wang, and Long Wang. “Fuzzy-Logic-Based Terrain Identification with Multisensor Fusion for Transtibial Amputees.” IEEE/ASME Transactions on Mechatronics IEEE/ASME Trans. Mechatron. 20.2 (2015): 618-30.
22. Tkach, D. C., and L. J. Hargrove. “Neuromechanical Sensor Fusion Yields Highest Accuracies in Predicting Ambulation Mode Transitions for Trans-tibial Amputees.” 2013 35th Annual International Conference of the IEEE Engineering in Medicine and Biology Society (EMBC) (2013).
23. Huang, He, Fan Zhang, L. J. Hargrove, Zhi Dou, D. R. Rogers, and K. B. Englehart. “Continuous Locomotion-Mode Identification for Prosthetic Legs Based on Neuromuscular-Mechanical Fusion.” IEEE Transactions on Biomedical Engineering IEEE Trans. Biomed. Eng. 58.10 (2011): 2867-875.
24. Rouse, E. J., Mooney, L. M., & Herr, H. M. “Clutchable Series-Elastic Actuator: Implications for Prosthetic Knee Design.” The International Journal of Robotics Research, 115 (2014).
25. D. A. Winter, “Biomechanical motor pattern in normal walking,” J. Mot. Behay., vol. 15, no. 4, pp. 302-330, 1983.
26. Au, Samuel K., and Hugh M. Herr. 2008. “Powered Ankle-Foot Prosthesis.” IEEE Robotics & Automation Magazine, no. September: 52-59.
27. Bellman, Ryan D., Matthew A. Holgate, and Thomas G. Sugar. 2008. “SPARKy 3: Design of an Active Robotic Ankle Prosthesis with Two Actuated Degrees of Freedom Using Regenerative Kinetics.” Proceedings of the 2nd Biennial IEEE/RAS-EMBS International Conference on Biomedical Robotics and Biomechatronics, BioRob 2008, 511-16.
28. Collins, Steven H., Myunghee Kim, Tianjian Chen, and Tianyao Chen. 2015. “An Ankle-Foot Prosthesis Emulator with Control of Plantarflexion and Inversion-Eversion Torque.” Proceedings—IEEE International Conference on Robotics and Automation 2015—June (June): 1210-16.
29. Olesnavage, Kathryn M., and Amos G. Winter. 2015. “Lower Leg Trajectory Error: A Novel Optimization Parameter for Designing Passive Prosthetic Feet.” IEEE International Conference on Rehabilitation Robotics 2015—September: 271-76.
30. Collins, Steven H., and Arthur D. Kuo. 2010. “Recycling Energy to Restore Impaired Ankle Function during Human Walking.” PLoS ONE 5 (2).
31. Duval, Jean Francois, and Hugh M. Herr. 2016. “FlexSEA: Flexible, Scalable Electronics Architecture for Wearable Robotic Applications.” Proceedings of the IEEE RAS and EMBS International Conference on Biomedical Robotics and Biomechatronics (2016) July: 1236-41.
U.S. Pat. No. 4,956,628 dated Sep. 11, 1990
U.S. Pat. No. 9,622,537 B2 dated Apr. 18, 2017
U.S. Pat. No. 7,171,331 B2 dated Jan. 30, 2007
U.S. Publication 2005/0107889 A1 dated May 19, 2005
WO 2011/000542 A1 dated Jan. 6, 2011
WO 2017/120063A1 dated Jul. 13, 2017

The relevant teachings of all patents, published applications and references cited herein are incorporated by reference in their entirety.

1. A method for controlling an autonomous wearable leg device, comprising the steps of:
a) collecting signals from one or more kinetic sensors embedded in a component of the autonomous wearable leg device
b) employing features extracted from these signals to predict or estimate gait events, gait activities, or terrains in real time
c) using predictions or estimates of gait events, gait activities, or terrains to send control commands to the autonomous wearable leg device in real time

2. The method of claim 1, where the autonomous wearable leg device is an autonomous transfemoral or transtibial prosthesis, exoskeleton, or orthotic device.

3. The method of claim 1, wherein one or multiple kinetic sensors are a load cell embedded within the prosthetic thigh or shank of a transfemoral or transtibial prosthesis.

4. The method of claim 1, wherein one or multiple kinetic sensors are embedded within a component of the autonomous wearable leg device support area.

5. The method of claim 4, wherein components of the autonomous wearable leg device support area include the prosthetic foot or foot cover, sock, insole, or shoe.

6. The method of claim 1, wherein gait events include establishment or breaking of physical contact between the foot and the ground, establishment of a foot-flat position, heel strike, toe strike, stopping and starting gait, and changing of gait speed.

7. The method of claim 1, wherein gait activities include walking, shuffling, jogging, or running in an anterior, posterior, or lateral direction, jumping, standing on the toes, or squatting.

8. The method of claim 1, wherein terrains include ascending or descending stairs, inclines, declines, obstacles, soft surfaces, and uneven surfaces.

9. The method of claim 1, further including the step of employing the kinetic signals to provide feedback to the user of the autonomous wearable leg device in real time.

10. The method of claim 9, wherein the feedback is in the form of at least one of mechanical stimuli and electrical stimuli.

11. The method of claim 10, wherein the feedback is in the form of mechanical stimuli.

12. The method of claim 11, wherein the mechanical stimuli include at least one member from the group consisting of: vibration; application of pressure to skin; pinching of skin; application of strain to skin; and variation of surface temperature applied to skin.

13. The method of claim 10, wherein the feedback is in the form of electrical stimuli.

14. The method of claim 13, wherein the electrical stimuli include at least one member selected from the group consisting of electrical stimulation of at least one of a muscle and a nerve.

15. A method for controlling an autonomous wearable leg device, comprising the steps of:
a) detecting or characterizing obstacles or terrain changes in real time using non-contact sensors integrated into a foot covering
b) using the detection or characterization of obstacles or terrains to modulate a control algorithm of the autonomous wearable leg device in real time

16. The method of claim 15, wherein the autonomous wearable leg device is a powered prosthesis, orthosis, or exoskeleton.

17. The method of claim 15, wherein the foot covering is a prosthetic cosmesis or a shoe.

18. The method of claim 15, wherein the non-contact sensors include one or multiple cameras, distance-measuring sensors, or laser scanners.

19. The method of claim 15, wherein one non-contact sensor is positioned in a forward orientation at a toe area of a shoe, whereby it is used to predict that the user will ascend stairs or clear an obstacle.

20. The method of claim 15, wherein one non-contact sensor is positioned in a downward orientation at the toe area of a shoe, whereby it is used to predict that the user will descent stairs when they position a lower extremity at the edge of a stair.

21. The method of claim 15, wherein one non-contact sensor is positioned in a backward orientation at the back area of a shoe, whereby it is used to detect stair descent.

22. The method of claim 15, wherein the control algorithm is used to actuate a prosthetic joint with a torque or position profile consistent with established biological norms for a predicted terrain.

23. An autonomous wearable leg device, comprising:
a) an ankle frame;
b) a pair of actuators, each actuator being mounted on the frame and connectable to a power source, and control signal, wherein the actuators are independently controllable;
c) an eccentric crank-arm linkage connecting the actuator to the foot interface;
d) a foot interface connected to the actuators, whereby actuation of either of the actuators transmits force to the foot interface; and
e) at least one hinge at the frame linking the ankle frame to the foot interface, whereby synchronous movement of the actuators causes plantarflexion or dorsiflexion of the foot interface component, and differential movement of the linkages causes eversion or inversion of the foot interface.

24. The autonomous device of claim 23, wherein the hinge includes a ball and socket assembly.

25. The autonomous device of claim 23, wherein the hinge includes a gimbal assembly.

26. The autonomous device of claim 24, wherein the gimbal assembly defines two axes of rotation, whereby one of the two axes defines an axis of rotation for dorsiflexion and plantarflexion, and the other of the two axes defines an axis of rotation for eversion and inversion of the first foot interface.

27. The autonomous device of claim 25, wherein the axes of rotation intersect.

28. The autonomous device of claim 26, wherein the axes of rotation intersect orthogonally.

29. The autonomous device of claim 26, wherein the axes of rotation intersect at an angle that is not orthogonal.

30. The autonomous device of claim 25, wherein the axes of rotation do not intersect.

31. The autonomous device of claim 29, wherein the axes of rotation are oriented orthogonally.

32. The autonomous device of claim 29, wherein the axes of rotation are oriented askew.

33. The autonomous device of claim 23, wherein the hinges include an ankle joint and subtalar joint linked to the ankle joint, wherein the ankle joint includes one degree of freedom, and the subtalar joint includes the other degree of freedom.

34. The autonomous device of claim 23, wherein the actuators are mirrored across a sagittal plane of the prosthesis.

35. The autonomous device of claim 23, wherein the actuators are each connected to the foot frame by way of an eccentric crank-arm linkage.

36. The autonomous device of claim 23, wherein the eccentric crank-arm linkage assembly of each actuator includes a gear reduction component having a serially-connected multi-stage timing belt drive-train and an output timing pulley linking the timing belt drive train with the respective linkage.

37. The autonomous device of claim 32, further including a foot component connected to the foot interface.

38. The autonomous device of claim 33, wherein the motors are split phase sector motors.

39. The autonomous device of claim 34, wherein the foot component includes at least one powered metatarsophalangeal joint.

40. The autonomous device of claim 23, further including a knee component connected to the ankle frame, having a single degree of freedom.

41. The autonomous device of claim 38, wherein the knee component includes a knee joint and a powered knee actuation system linked to the knee joint, wherein the knee joint has the degree of freedom of the knee component.

42. The autonomous device of claim 39, wherein the powered knee actuation system includes a clutched series static actuator.

43. The autonomous device of claim 40, wherein ankle component includes an ankle joint and a subtalar joint linked to the ankle joint, wherein the ankle joint includes one degree of freedom, and the subtalar joint includes the other degree of freedom of the ankle component.

44. The autonomous device of claim 41, wherein the foot component includes a metatarsophalangeal joint having the degree of freedom of the foot component.

45. The autonomous device of claim 42, wherein the knee component further includes a series elastic actuator.

46. The autonomous device of claim 43, wherein the series elastic actuator is a clutchable series elastic actuator.

47. A foot device that functions as one or more powered metatarsophalangeal joints, comprising:
a) a mounting plate that is a foot frame;
b) an actuator mounted on the foot frame and connectable to a power source, and control signal, wherein the actuator is independently controllable; and
c) a hinge linked to the foot frame, and
d) a toe component that is linked to the actuator, whereby the actuator causes the toe component to move relative to the hinge joint, thereby functioning as a powered metatarsophalangeal joint of the foot.

48. The foot device of claim 47, wherein the hinge is a flexural hinge.

49. The foot device of claim 47, wherein the hinge is a revolute hinge.

50. The foot device of claim 47, further including a parallel elastic element with one end fixed to the foot frame and the other end free to apply force to the toe component about the hinge, whereby elastic force of the elastic element moves the hinge to a minimum energy position.

51. The foot device of claim 47, further including an appendage component assembly having parallel elastic members.

52. The foot device of claim 51, wherein the parallel elastic members are one or a plurality of cantilever beams fixed to the foot frame and apply a normal force a distance from the hinge toward the toe component.

53. The foot device of claim 51, further including rigid spring clamps, wherein the parallel elastic members are fixed to the foot frame by being clamped between the rigid spring clamps and the foot frame.

54. The foot device of claim 51, wherein the actuator includes a motor, a capstan, a tensile strand.

55. The foot device of claim 54 wherein the stiffness of the hinge is modulated by the change in length of the tensile strand affecting the preload of the parallel elastic members.

56. The foot device of claim 47, wherein the hinge includes a parallel elastic element and at least one of a crank arm, a push-rod assembly, and an actuator that provides output torque regulation by modulating the motion of the output appendage about the joint and thereby modulating the loading of the parallel elastic element.

57. A method for controlling an autonomous wearable device, comprising the steps of:
a) collecting kinetic signals from an array of sensors embedded in a prosthetic, orthotic or exoskeletal component of the autonomous wearable device worn by a subject during a portion of a gait cycle;
b) extracting raw values of at least one feature from the collected kinetic signals;
c) applying the raw values of the feature to a controller;
d) generating a controlling command with the controller; and
e) sending the controlling command to the prosthetic, orthotic or exoskeletal component to thereby control the prosthetic, orthotic or exoskeletal component during the portion of the gait cycle.

58. The method of claim 57, further including classifying the raw values according to a classification algorithm or normalizing the raw values before being applying the raw values to the controller.

59. The method of claim 58, wherein the normalization is by at least one member of the group consisting of: feature mean and standard deviation; and feature extreme and range.

60. The method of claim 57, further including the step of compounding the at least one feature.

61. The method of claim 60, wherein the raw values of a plurality of features are extracted.

62. The method of claim 61, wherein the plurality of features are compounded to produce a secondary feature.

63. The method of claim 58 wherein the at least one feature includes at least one member of the group consisting of: a statistical feature; a time domain feature; a frequency domain feature; and combinations thereof.

64. The method of claim 63, wherein the at least one feature includes a statistical feature.

65. The method of claim 64, wherein the statistical feature includes at least one member of the group consisting of: mean; maximum; minimum; range; mean absolute deviation; variance; standard deviation; and sample size.

66. The method of claim 65, wherein the statistical feature is extracted from the kinetic signals generated during a stance phase of the gait cycle.

67. The method of claim 66, further including the step of detecting the kinetic signals by thresholding an estimated ground reaction force.

68. The method of claim 63, wherein the at least one feature includes a time domain feature.

69. The method of claim 68, wherein the at least one time domain feature is employed in an ARIMA model included in a classification algorithm.

70. The method of claim 69, wherein the at least one time domain feature is selected from the group consisting of: minimum and maximum values calculated from at least one of a beginning and end of a signal collection period; a difference between a combination of the beginning and end of the signal collection period; a number of zero or mean crossings; cross correlations between signals; signal autocorrelations; parameters or orders of autoregressive, integrated, and moving average parts; and combinations thereof.

71. The method of claim 70, wherein the at least one time domain feature is normalized by a length of a stance period during the gait cycle.

72. The method of claim 63, wherein the at least one feature includes a frequency domain feature.

73. The method of claim 63, further including classifying the raw values according to a classification algorithm.

74. The method of claim 72, wherein the at least one frequency domain feature includes: frequencies of k peaks in amplitude in a discrete Fourier transform (DFT) of the collected kinetic signals; kth quartiles of the DFT; and total signal power.

75. The method of claim 57, wherein control of the autonomous wearable device further includes: the step of triggering at least one transition between two members selected from the group consisting of: walking; jogging; running; standing; and sitting.

76. The method of claim 75, wherein the at least one transition employs only the kinetic signals.

77. The method of claim 76, wherein the triggering includes thresholding a vertical ground reaction force.

78. The method of claim 77, wherein the triggering step includes distinguishing at least one member of the group consisting of: walking; jogging; running; and between combinations thereof.

79. The method of claim 78, wherein the triggering step includes distinguishing between at least two members of the group consisting of: walking; running; and jogging.

80. The method of claim 79, wherein the step of distinguishing between the at least two members of the group consisting of: running, walking, and jogging, includes determining the frequency or amplitude of ground reaction force fluctuation.

81. The method of claim 80, wherein the step of distinguishing between the at least two members of the group consisting of: running, walking, or jogging, includes anticipating terrain and transitioning in real time.

82. The method of claim 81, wherein the step of anticipating terrain includes the step of pattern recognition among the collected kinetic signals during a phase of the gait angle.

83. The method of claim 82, wherein the pattern recognition step includes the at least one kinetic signal in at least one member selected from the group consisting of: a Naïve Bayesian classification; a decision tree classification; a discriminant analysis classifier; a referral network classifier; and a logistic regression classifier.

84. The method of claim 57, wherein the array of kinetic sensors is located at at least a portion of a sole of the prosthetic, orthotic or exoskeletal component.

85. The method of claim 84, wherein the array of kinetic sensors is located at a portion of the sole of the prosthetic, orthotic or exoskeletal component.

86. The method of claim 85, wherein the portion of the sole is at least one member of the group consisting of a heel, an arch, a ball region, and a center of pressure of the prosthetic, orthotic or exoskeletal component.

87. The method of claim 84, wherein the array of kinetic sensors is embedded across essentially the entire sole of the prosthetic, orthotic or exoskeletal component.

88. The method of claim 84, further including the step of employing at least one biomimetic control profile to control at least one control variable of the prosthetic, orthotic, or exoskeletal component in response to pattern recognition produced by the pattern recognition step.

89. The method of claim 88, wherein a plurality of biomimetic control profiles are employed.

90. The method of claim 89, wherein the biomimetic control profiles employed correspond to at least one state of at least one task type.

91. The method of claim 90, wherein the task type includes at least one member selected from the group consisting of: walking on a level or sloped terrain; walking upstairs; walking downstairs; jogging on level or sloped terrain; jogging upstairs; jogging downstairs; running on level or slopped terrain; running upstairs; and running downstairs.

92. The method of claim 85, wherein the at least one control variable of the at least one biomimetic control profile includes at least one member selected from the group consisting of: center pressure; radial power; linear power; force; position; velocity; and work.

93. The method of claim 92, further including the step of modulating at least one control variable in accordance with at least one of a threshold and a calculation employing at least one state variable of at least one task type.

94. The method of claim 93, wherein the at least one state variable includes at least one member selected from the group consisting of: at least a portion of the collected kinetic signals; joint kinetics; ambulatory speed; user intact; variation of ground surface slope, altitude; material; added mechanical loads on the user; and user posture.

95. The method of claim 57, further including employing the kinetic signals to generate a data base.

96. The method of claim 95, further including the kinetic signals of the data base to thereby produce diagnostic information from at least one member of the group consisting of: diagnostic statistics; step counts; information about power, information about force output; time spent; electrical work done; and metabolic work done.

97. The method of claim 96, wherein the diagnostic information is directed to at least one member selected from the group consisting of modes, tasks and status.

98. The method of claim 57, further including the step of employing the kinetic signals to provide feedback to the subject.

99. The method of claim 98, wherein the feedback is in the form of at least one of mechanical stimuli and electrical stimuli.

100. The method of claim 99, wherein the feedback is in the form of mechanical stimuli.

101. The method of claim 100, wherein the mechanical stimuli include at least one member from the group consisting of: vibration; application of pressure to skin; pinching of skin; application of strain to skin; and variation of surface temperature applied to skin.

102. The method of claim 99, wherein the feedback is in the form of electrical stimuli.

103. The method of claim 102, wherein the electrical stimuli include at least one member selected from the group consisting of electrical stimulation of at least one of a muscle and a nerve.

104. A method for controlling an autonomous wearable leg device, comprising the steps of:
a) using non-contact sensors integrated into a foot covering to detect or characterize obstacles or terrain changes in real time; and
b) using the detection or characterization of obstacles or terrains to modulate a control algorithm of the autonomous wearable leg device in real time

105. The method of claim 104, wherein the autonomous wearable leg device is a powered prosthesis, orthosis, or exoskeleton.

106. The method of claim 105, wherein the foot covering is a prosthetic cosmesis or a shoe.

107. The method of claim 106, wherein the non-contact sensors include one or multiple cameras, distance-measuring sensors, or laser scanners.

108. The method of claim 104, wherein one non-contact sensor is positioned in a forward orientation at a toe area of a shoe, whereby it is used to predict that the user will ascend stairs or clear an obstacle.

109. The method of claim 105, wherein one non-contact sensor is positioned in a downward orientation at the toe area of a shoe, whereby it is used to predict that the user will descent stairs when they position a lower extremity at the edge of a stair.

110. The method of claim 105, wherein one non-contact sensor is positioned in a backward orientation at the back area of a shoe, whereby it is used to detect stair descent.

111. The method of claim 105, wherein the control algorithm is used to actuate a prosthetic joint with a torque or position profile consistent with established biological norms for a predicted terrain.

112. A autonomous wearable leg device for integrated, real time, and kinetic sensing, comprising:
a) a prosthetic, orthotic or exoskeletal component that includes a support area;
b) an array of sensors embedded along the support area; and
c) a controller in communication with the array, whereby the sensors of the array can collectively sense and transmit spatially-dependent pressure signals to the controller, and whereby the controller can generate a controlling command and send the controlling command to the prosthetic, orthotic or exoskeletal component, and thereby control the prosthetic, orthotic, or exoskeletal component.

113. The device of claim 112, wherein the component includes at least one member of the group consisting of: a prosthetic foot; a foot cover; a prosthetic socket; a sole of a shoe; a sock; a six-axis load cell; a transtibial load cell; and a transfemoral load cell.

114. The device of claim 112, wherein the sensors are arrayed to estimate, in combination with the controller, at least one of a center of pressure and strain.

115. The device of claim 112, wherein the sensors are arrayed in a transverse plane to a longitudinal axis of a subject wearing the device.

116. The device of claim 112, wherein the sensors are arrayed to estimate, in combination with the controller, a ground reaction force on the array.

117. The device of claim 112, wherein the component is a transverse foot section and the sensors are arrayed to estimate, in combination with the controller, a ground reaction force on the transverse foot section.

118. The device of claim 117, wherein the transverse foot section is at least one member of the group consisting of a ball, a heel, an arch, and a combination thereof.

119. An autonomous prosthetic, orthotic, or exoskeletal device, comprising:
a) an ankle frame;
b) a pair of actuators, each actuator being mounted on the frame and connectable to a power source, and control signal, wherein the actuators are independently controllable;
c) a foot interface connected to the actuators, whereby actuation of either of the actuators transmits force to the foot interface; and
d) at least one hinge at the frame linking the ankle frame to the foot interface, whereby synchronous movement of the actuators causes plantarflexion or dorsiflexion of the foot interface component, and differential movement of the linkages causes eversion or inversion of the foot interface.

120. The autonomous device of claim 119, wherein the hinge includes a gimbal assembly.

121. The autonomous device of claim 120, wherein the gimbal assembly defines two axes of rotation, whereby one of the two axes defines an axis of rotation for dorsiflexion and plantarflexion, and the other of the two axes defines an axis of rotation for eversion and inversion of the first foot interface.

122. The autonomous device of claim 121, wherein the axes of rotation intersect.

123. The autonomous device of claim 122, wherein the axes of rotation intersect orthogonally.

124. The autonomous device of claim 122, wherein the axes of rotation intersect at an angle that is not orthogonal.

125. The autonomous device of claim 121, wherein the axes of rotation do not intersect.

126. The autonomous device of claim 125, wherein the axes of rotation are oriented orthogonally.

127. The autonomous device of claim 125, wherein the axes of rotation are oriented askew.

128. The autonomous device of claim 119, wherein the at least one hinge includes an ankle joint and subtalar joint linked to the ankle joint, wherein the ankle joint includes one degree of freedom, and the subtalar joint includes the other degree of freedom.

129. The autonomous device of claim 119, wherein the actuators are mirrored across a sagittal plane of the prosthesis.

130. The autonomous device of claim 119, wherein each actuator includes a gear reduction component having a serially-connected multi-stage timing belt drive-train and an output timing pulley linking the timing belt drive train with the respective linkage.

131. The autonomous device of claim 128, further including a foot component connected to the foot interface.

132. The autonomous device of claim 119, wherein the actuators are split-phase sector motors.

133. The autonomous device of claim 132, wherein the foot component includes a powered metatarsophalangeal joint.

134. The autonomous device of claim 119, further including a knee component connected to the ankle frame, having a single degree of freedom.

135. The autonomous device of claim 134, wherein the knee component includes a knee joint and a powered knee actuation system linked to the knee joint, wherein the knee joint has the degree of freedom of the knee component.

136. The autonomous device of claim 135, wherein the powered knee actuation system includes a clutched series static actuator.

137. The autonomous device of claim 136, wherein ankle component includes an ankle joint and a subtalar joint linked to the ankle joint, wherein the ankle joint includes one degree of freedom, and the subtalar joint includes the other degree of freedom of the ankle component.

138. The autonomous device of claim 137, wherein the foot component includes a metatarsophalangeal joint having the degree of freedom of the foot component.

139. The autonomous device of claim 138, wherein the knee component further includes a series elastic actuator.

140. The autonomous device of claim 139, wherein the series elastic actuator is a clutchable series elastic actuator.

141. A prosthetic foot that functions as a powered metatarsophalangeal joint, comprising:
a) a mounting plate that is a foot frame;
b) an actuator mounted on the foot frame and connectable to a power source, and control signal, wherein the actuator is independently controllable; and
c) a hinge linked to the foot frame, and
d) a toe component that is linked to the actuator, whereby the actuator causes the toe component to move relative to the hinge joint, thereby functioning as a powered metatarsophalangeal joint of the foot.

142. The prosthetic foot of claim 141, wherein the hinge is a flexural hinge.

143. The prosthetic foot of claim 141, wherein the hinge is a revolute hinge.

144. The prosthetic foot of claim 141, further including a parallel elastic element with one end fixed to the foot frame and the other end free to apply force to the toe component about the hinge, whereby elastic force of the elastic element moves the hinge to a minimum energy position.

145. The prosthetic foot of claim 141, further including a foot component assembly at the parallel elastic element.

146. The prosthetic foot of claim 145, wherein the parallel elastic element includes one or a plurality of cantilever beams fixed to the foot frame and apply a normal force a distance from the hinge toward the toe component.

147. The prosthetic foot of claim 145, further including rigid spring clamps, wherein the parallel elastic members are fixed to the foot frame by being clamped between the rigid spring clamps and the foot frame.

148. The prosthetic foot of claim 141, wherein the actuator includes a motor, a capstan, a tensile strand.

149. The prosthetic foot of claim 141, wherein the stiffness of the hinge is modulated by the change in length of the tensile strand affecting the preload of the parallel elastic members.

150. The prosthetic foot of claim 141, wherein the hinge includes a parallel elastic element and at least one of a crank arm, a push-rod assembly, and an actuator at the hinge joint that provides output torque regulation by modulating the loading of the parallel elastic element.

151. An autonomous prosthesis, comprising:
a) an actuated knee component with a single actuated degree of freedom;
b) an ankle component with two actuated degrees of freedom and linked to the actuated knee component; and
c) a foot component with a single actuated degree of freedom and linked to the ankle component.

152. An autonomous prosthesis, comprising:
a) an actuated knee component with a single actuated degree of freedom;
b) an ankle component with two actuated degrees of freedom and linked to the actuated knee component; and
c) a passive foot component.

153. An autonomous prosthesis, comprising:
a) a passive knee component;
b) an ankle component with two actuated degrees of freedom and linked to the passive knee component; and
c) a foot component with a single actuated degree of freedom and linked to the ankle component.

154. An autonomous prosthesis, comprising:
a) a passive knee component;
b) an ankle component with two actuated degrees of freedom and linked to the passive knee component; and
c) a passive foot component linked to the ankle component.

155. An autonomous prosthesis, comprising:
a) an ankle component with two actuated degrees of freedom; and
b) a foot component with a single actuated degree of freedom and linked to the ankle component.

156. An autonomous prosthesis, comprising:
a) an ankle component with two actuated degrees of freedom; and
b) a passive foot component linked to the ankle component.

157. A prosthetic device that functions as a powered metatarsophalangeal joint, comprising:
a) a mounting plate that is a frame;
b) an actuator mounted on the frame and connectable to a power source, and a control signal, wherein the actuator is independently controllable;
c) a hinge linked to the frame; and
d) an appendage that is linked to the actuator, whereby the actuator causes the appendage to move relative to the hinge joint.

158. The prosthetic device of claim 157, wherein the hinge is a flexural hinge.

159. The prosthetic device of claim 157, wherein the hinge is a revolute hinge.

160. The prosthetic device of claim 157, further including a parallel elastic element with one end fixed to the frame and the other end free to apply force to the appendage about the hinge, whereby elastic force of the elastic element moves the hinge to a minimum energy position.

161. The prosthetic device of claim 157, further including an appendage component assembly having parallel elastic members.

162. The prosthetic device of claim 157, wherein the parallel elastic members are one or a plurality of cantilever beams fixed to the foot frame and apply a normal force a distance from the hinge toward the appendage.

163. The prosthetic device of claim 162, further including rigid spring clamps, wherein the parallel elastic members are fixed to the frame by being clamped between the rigid spring clamps and the frame.

164. The prosthetic device of claim 157, wherein the actuator includes a motor, a capstan, a tensile strand.

165. The prosthetic device of claim 164, wherein the stiffness of the hinge is modulated by the change in length of the tensile strand affecting the preload of the parallel elastic members.

166. The prosthetic device of claim 157, wherein the hinge includes a parallel elastic element and at least one of a crank arm, a push-rod assembly, and an actuator at the hinge joint that provides output torque regulation by modulating the loading of the parallel elastic element.
